(19) United States
(12) Patent Application Publication
(10) Pub. No.: US 2020/0384104 A1
(43) Pub. Date: Dec. 10, 2020

(54) Structure-Function Relationships in the Development of Immunotherapeutic Agents

(71) Applicant: NORTHWESTERN UNIVERSITY, Evanston, IL (US)

(72) Inventors: Chad A. Mirkin, Wilmette, IL (US); Shuya Wang, Evanston, IL (US); Bin Zhang, Chicago, IL (US); Kacper Skakuj, Durham, NC (US)

(21) Appl. No.: 16/772,551

(22) PCT Filed: Dec. 14, 2018
(86) PCT No.: PCT/US18/65765

§ 371 Date: Jun. 12, 2020

Related U.S. Application Data
(60) Provisional application No. 62/599,395, filed on Dec. 15, 2017.

(51) Int. Cl. A61K 39/39; A61P 35/00; A61K 39/385; A61K 9/127; A61K 39/00; G01N 33/50

(52) U.S. Cl. CPC A61K 39/39; A61P 35/00; A61K 39/385; A61K 9/127; A61K 2039/55555; G01N 33/5047; A61K 2039/55561; A61K 2039/627; A61K 39/0011

(57) ABSTRACT

The present disclosure provides compositions and methods comprising spherical nucleic acid (SNA) components for use as immunotherapeutic agents. The disclosure provides a method comprising: treating a population of antigen presenting cells with a SNA comprising a nanoparticle, an antigen, and an adjuvant; and determining a time at which the population of antigen presenting cells presents a maximal signal that is indicative of antigen presentation by the antigen presenting cells and a time at which the population of antigen presenting cells presents a maximal co-stimulatory signal due to the adjuvant. The disclosure includes compositions that comprise a pharmaceutically acceptable carrier and a SNA of the disclosure, wherein the SNA comprises a nanoparticle, an oligonucleotide on the surface of the nanoparticle, and an antigen that is associated with the surface of the SNA via a linker. The disclosure additionally includes articles of manufacture and kits.

### CROSS REFERENCE TO RELATED APPLICATIONS

This application claims the priority benefit under 35 U.S.C. § 119(e) of U.S. Provisional Patent Application No. 62/599,395, filed Dec. 15, 2017, the disclosure of which is incorporated herein by reference in its entirety.

### STATEMENT OF GOVERNMENT INTEREST

This invention was made with government support under U54 CA199091 awarded by the National Institutes of Health, and N00014-15-1-0043 awarded by the Office of Naval Research. The government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 2017-215_Seqlisting.txt; Size: 3,433 bytes; Created: Dec. 14, 2018), which is incorporated by reference in its entirety.

### BACKGROUND

Fighting cancer through immunotherapy, by engaging and steering a patient's immune system to attack cancer cells, is a powerful therapeutic approach¹⁻³. In particular, the success of adoptive cell transfer (ACT) strategies and checkpoint inhibitors (targeting PD-1, PD-L1, CTLA4), especially for treating melanoma and lung cancer, have revealed the power of unlocking the immune system to attack tumors⁴⁻⁶. Indeed, a dramatic response to checkpoint inhibitors in a subset of patients with advanced cancer has been documented. In addition to such approaches, injectable vaccines are particularly attractive because, in principle, they do not involve cell harvesting and thereby provide a convenient, safe, and low-cost way to boost a patient's immune system^{7,8}.

A major challenge in the development of vaccines is the design and selection of the vehicle for delivering adjuvant and antigen molecules¹. In principle, as with any therapeutic, the structure could have a significant influence on safety, efficacy, and potency^{9,10}. In the case of vaccines, the way multiple molecular components are formulated could have a major influence on bio-distribution and delivery to cells of the immune system, and on the activation of immunostimulatory pathways that ultimately lead to the priming and expansion of antigen-specific T-cells^{11,12}.

### SUMMARY

In the case of cancer immunotherapy, nanostructures are attractive because they can carry all of the necessary components of a vaccine, including both antigen and adjuvant. Herein, spherical nucleic acids (SNAs), an emerging class of nanotherapeutic materials, are provided that can be used to, in various aspects, deliver peptide antigens and nucleic acid adjuvants to raise immune responses that, in various embodiments, kill cancer cells and reduce (or eliminate) tumor growth.

Accordingly, in some aspects the disclosure provides a method comprising: treating a population of antigen presenting cells with a spherical nucleic acid (SNA) comprising a nanoparticle, an antigen, and an adjuvant; and determining a time at which the population of antigen presenting cells presents a maximal signal that is indicative of antigen presentation by the antigen presenting cells and a time at which the population of antigen presenting cells presents a maximal co-stimulatory signal due to the adjuvant. In some embodiments, the antigen presenting cells are lymphocytes or dendritic cells (DCs). In some embodiments, one adjuvant or antigen is employed (i.e., only one type of adjuvant is present). Alternatively, more than one adjuvant or antigen (e.g., two, three, four, five, or more different adjuvants or antigens) are used.

In further aspects, the disclosure provides a method of selecting a spherical nucleic acid (SNA) for increased ability to activate antigen presenting cells, comprising: generating a first SNA comprising a nanoparticle, an antigen, and an adjuvant and a second SNA comprising nanoparticle, an antigen, and an adjuvant; treating a first population of antigen presenting cells with the first SNA and treating a second population of antigen presenting cells with the second SNA; determining a time at which the first population of antigen presenting cells presents a maximal signal that is indicative of antigen presentation and a time at which the first population of antigen presenting cells presents a maximal co-stimulatory signal due to the adjuvant; determining a time at which the second population of antigen presenting cells presents a maximal signal that is indicative of antigen presentation and a time at which the second population of antigen presenting cells presents a maximal co-stimulatory signal due to the adjuvant; and selecting as the SNA for which time to achieve maximal signal for antigen presentation is the same as or about the same as time to achieve maximal co-stimulatory signal. In some embodiments, the antigen presenting cells or lymphocytes or dendritic cells. In some embodiments, one adjuvant or antigen is employed (i.e., only one type of adjuvant is present). Alternatively, more than one adjuvant or antigen (e.g., two, three, four, five, or more different adjuvants or antigens) are used.

In some aspects, a spherical nucleic acid (SNA) is provided, comprising a nanoparticle, an adjuvant, and an antigen, wherein: the adjuvant comprises an oligonucleotide comprising an immunostimulatory nucleotide sequence and an associative moiety that allows association of the immunostimulatory sequence with the nanoparticle; and the antigen is attached to the nanoparticle through a linker. In some embodiments, one adjuvant or antigen is employed (i.e., only one type of adjuvant is present). Alternatively, more than one adjuvant or antigen (e.g., two, three, four, five, or more different adjuvants or antigens) are used.

In some embodiments, the immunostimulatory nucleotide sequence is a toll-like receptor (TLR) agonist. In further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the immunostimulatory nucleotide sequence comprises a CpG nucleotide sequence.

In some embodiments, the linker is a carbamate alkylene disulfide linker. In further embodiments, the antigen is attached to the nanoparticle through the linker according to Antigen-NH—C(O)—O-C₂₋₅alkylene-S—S-C₂₋₇alkylene, or Antigen-NH—C(O)—O-CH2-Ar—S—S-C₂₋₇alkylene, wherein Ar comprises a meta- or para-substituted phenyl. In some embodiments, the antigen is attached to the nanoparticle through the linker according to Antigen-NH—C(O)—O-C₂₋₄alkylene-C(W)(X)—S—S—CH(Y)(Z)C₂₋₆alkylene, and W and X, Y and Z are each independently H, Me, Et, or iPr. In further embodiments, the antigen is attached to the nanoparticle through the linker according to Antigen-NH—C(O)—O—CH₂—Ar—S—S—CX(Y)C₂₋₆alkylene, and X and Y are each independently Me, Et, or iPr.

In some embodiments, the linker is an amide alkylene disulfide linker. In further embodiments, the antigen is attached to the nanoparticle through the linker according to Antigen-NH—C(O)-C₂₋₅alkylene-S—S-C₂₋₇alkylene. In further embodiments, the antigen is attached to the nanoparticle through the linker according to Antigen-NH—C(O)—C(W)(X)C₂₋₄alkylene-S—S—CH(Y)(Z)C₂₋₆alkylene, and W and X, Y and Z are each independently H, Me, Et, or iPr.

In some embodiments, the linker is a amide alkylene thio-succinimidyl linker. In further embodiments, the antigen is attached to the nanoparticle through the linker according to Antigen-NH—C(O)-C₂₋₄alkylene-N-succinimidyl-S-C₂₋₆alkylene.

In some embodiments, the antigen is a tumor associated antigen, a tumor specific antigen, a neo-antigen. In further embodiments, the antigen is OVA1, MSLN, P53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, or human papillomavirus (HPV) E7 nuclear protein.

In some embodiments, the nanoparticle is a liposome. In further embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl -sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

In some embodiments, the associative moiety is tocopherol, cholesterol, 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-di-(9Z-octadecenoyl)-sn-glycero-3-phosphoethanolamine (DOPE), or 1,2-dihexadecanoyl-sn-glycero-3-phosphoethanolamine (DPPE).

In further embodiments, the adjuvant comprises RNA or DNA. In still further embodiments, the adjuvant comprises an agonist of an innate immune system signal pathway member (e.g., GM-CSF, PAMP receptor agonist). In some embodiments, the adjuvant comprises Freund's adjuvant. The disclosure contemplates use of more than one type of adjuvant.

In some embodiments, a SNA of the disclosure further comprises an additional oligonucleotide. In some embodiments, the additional oligonucleotide comprises RNA or DNA. In further embodiments, said RNA is a non-coding RNA. In still further embodiments, said non-coding RNA is an inhibitory RNA (RNAi). In some embodiments, the RNAi is selected from the group consisting of a small inhibitory RNA (siRNA), a single-stranded RNA (ssRNA) that forms a triplex with double stranded DNA, and a ribozyme. In further embodiments, the RNA is a microRNA. In some embodiments, said DNA is antisense-DNA.

In some embodiments, the nanoparticle has a diameter of 50 nanometers or less. In further embodiments, a SNA of the disclosure comprises about 10 to about 200 (e.g., about 10 to about 80) double stranded oligonucleotides. In some embodiments, a SNA of the disclosure comprises 75 double stranded oligonucleotides. In further embodiments, a SNA of the disclosure comprises about 10 to about 200 (e.g., about 10 to about 80) single stranded oligonucleotides. In some embodiments, a SNA of the disclosure comprises 75 single stranded oligonucleotides. In some embodiments, a SNA comprises 0.1-100 pmol/cm³ oligonucleotides (double or single stranded) on the surface.

In various aspects, a SNA of the disclosure is contemplated for use according to any method described herein.

In some aspects, the disclosure provides a composition comprising a SNA as disclosed herein or obtained by a method as disclosed herein in a pharmaceutically acceptable carrier. In some embodiments, the composition is capable of generating an immune response in an individual upon administration to the individual. In further embodiments, the immune response comprises antibody generation or a protective immune response.

In some aspects, the disclosure provides a vaccine comprising a composition of the disclosure, and an adjuvant. In some aspects, the immune response is a neutralizing antibody response or a protective antibody response.

In some aspects, the disclosure provides a method of producing an immune response to cancer in an individual, comprising administering to the individual an effective amount of a composition or vaccine of the disclosure, thereby producing an immune response to cancer in the individual.

In further aspects a method of inhibiting expression of a gene is provided comprising hybridizing a polynucleotide encoding the gene with one or more oligonucleotides complementary to all or a portion of the polynucleotide, the oligonucleotide being an additional oligonucleotide as disclosed herein, wherein hybridizing between the polynucleotide and the oligonucleotide occurs over a length of the polynucleotide with a degree of complementarity sufficient to inhibit expression of the gene product. In some embodiments, expression of the gene product is inhibited in vivo. In some embodiments, expression of the gene product is inhibited in vitro.

In some aspects, the disclosure provides a method for up-regulating activity of a toll-like receptor (TLR) comprising contacting a cell having the TLR with a SNA of the disclosure, which is understood to include a SNA obtained by a method as described herein. In some embodiments, the adjuvant comprises a TLR agonist. In further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLRS), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the method is performed in vitro. In further embodiments, the method is performed in vivo. In some embodiments, the cell is an antigen presenting cell (APC). In further embodiments, the APC is a dendritic cell. In still further embodiments, the cell is a leukocyte. In some embodiments, the leukocyte is a phagocyte, an innate lymphoid cell, a mast cell, an eosinophil, a basophil, a natural killer (NK) cell, a T cell, or a B cell. In some embodiments, the phagocyte is a macrophage, a neutrophil, or a dendritic cell.

In some aspects, the disclosure provides a method of immunizing an individual against cancer comprising administering to the individual an effective amount of a composition of the disclosure, thereby immunizing the individual against cancer. In some embodiments, the composition is a cancer vaccine. In further embodiments, the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.

### BRIEF DESCRIPTION OF THE FIGURES

FIG. 1 depicts an evaluation of the dependence of CpG and antigen co-delivery on SNA structure. (A) Scheme of three designs of SNA-E, A and H. (B) Uptake of CpG (Cy5) and OVA1 (TMR) by BMDCs in vitro, measured by flow cytometry. (C) Fraction of cells showing high levels of both CpG and OVA1, recovered from the DLN of mice (n=3) 2 hours following subcutaneous injection with reagents as indicated, as determined by flow cytometry. Values are an average of three replicates. (D) Images of cells recovered from DLN from mice 4 hours following immunization by subcutaneous injection, visualized by confocal microscopy. OVA1 peptide labeled with TMR was shown in green and CpG labeled with Cy5 was shown in red. (E) The fluorescence intensity for OVA1 peptide and CpG of the images. (F) Subcellular co-localization of peptide and CpG was quantified by Mander's coefficient (values of r>0.6 indicate strong co-localization). Data presented as mean±SEM (B,C,E,F). ***P<0.001, **P<0.01, *P<0.05.

FIGS. 2A-2F shows (a) Mass-spectrum of Oligonucleotides and Oligonucleotide-peptide conjugates. MALDI-TOF spectrum of DNA oligonucleotides and DNA-peptide conjugates. Matrix: 2′,6′- dihydroxyacetophenone (DHAP) in negative linear mode. Expected masses of conjugates are 6650.45 Da (Comp. strand), 7716.73Da (Comp.+C-OVA1 peptide conjugation), 4151 (Anchored strand), and 5217.2 (Anchored strand+C-OVA1 peptide conjugation). MALDI-TOF results meet the range requirement of calculated mass. (b) Formation of duplex DNA with CpG and complementary oligonucleotide conjugated to peptide antigen. To form duplex DNA, equimolar mixtures of peptide-oligonucleotide conjugate and CpG-3′-cholesterol were prepared and in buffer (1× Duplex buffer, IDT) to a concentration of 200 μM. Mixtures were heated to 70° C. for 10 minutes, allowed to cool to room temperature and incubated at 4° C. overnight. Analysis by native PAGE gel electrophoresis (20% acrylamide, TBE buffer) showed the formation of duplex DNA and the absence of single stranded oligonucleotides (stained by SYBR Green II). (c) Dynamic Light Scattering of SNAs. The size of extruded liposome cores and of three SNA structures were analyzed by dynamic light scattering (DLS). The hydrodynamic diameters (DH) of the nanoparticles were calculated with Malvern Zetasizer software using the Stokes-Einstein equation (D_{H}=kBT/3πηD, where kB is the Boltzmann constant, T is the absolute temperature, and η is the solvent viscosity, and D is the diffusion constant obtained experimentally by fit). The polydispersity index (PDI) was calculated as the width of the size distribution using cumulants analysis, and had measured values of: Liposome: 0.074±0.009; SNA-E: 0.109±0.007; SNA-H: 0.098±0.005; SNA-A: 0.104±0.011. (d) Zeta potential of Liposome Cores and SNAs. Zeta potential measurements were performed to show change in surface charge of SNAs upon the adsorption of DNA and DNA-peptide conjugates to liposomes. Zeta potential decreased upon addition of DNA or DNA-peptide conjugates, indicating successful surface loading. Within all three SNAs structure, values of zeta potential (mV) are comparable: Liposome: −1.169±0.426; SNA-E: −20.38±1.270; SNA-A: −19.33±0.512; SNA-H: −22.43±0.531. (e) Cryo-EM of Liposomes and SNAs. To analyze the liposomal SNAs by cryo-EM, SNA samples were cast onto copper grids with lacey carbon using FEI Vitrobot Mark III. The grid was imaged using a Hitachi HT7700 TEM with Gatan cryo transfer holder. (f) Electrophoretic mobility of SNAs and the adsorption of ˜75 cholesterol-terminated oligonucleotides or duplexes per liposome. To examine the adsorption of DNA to liposomes in SNA preparation, 3′-cholesterol modified CpG oligonucleotide was added to aliquots of liposome solution and allowed to shake overnight, 37° C. Different ratios of DNA to liposome, ranging from 25:1 to 125:1 were used. SNAs were analyzed by electrophoresis (1% agarose) and staining by SYBR Green II (300 ng DNA per well). Analysis of the intensity of the bands in the gel is shown in the right panel (determined by ImageJ analysis).

FIG. 3 depicts an evaluation of time-dependent intracellular fate of antigens delivered by three SNAs structures by confocal microscopy. Images of OVA1 peptide (Cy5, red) co-localized with (A) late endosome (green, Rab9) or (B) ER (green, PDI) delivered by SNA-E, A and H. (C) Peptide intensity per cell over time. (D) Manders' overlap coefficient representing the fraction of endosomes where the Rab9 signal is co-localized with Cy5. (E) Manders' overlap coefficient representing the fraction of the ER where the PDI signal is co-localized with Cy5. SNA-H has a major advantage over SNA-A and SNA-E in the temporal release of antigen, by way of increased retention of peptide within the endosomes of BMDCs throughout the 24 hour period. All analysis values are an average of 10-15 random selected images. Data presented as mean±SEM (C,D,E). ***P<0.001, **P<0.01, *P<0.05.

FIG. 4 shows the kinetics of DC activation with SNAs. (A) Kinetics of antigen (OVA1) presentation and expression of co-stimulation marker (CD86) by BMDCs upon treatment with SNAs, determined by flow cytometry. (B) Number of DLN cells from mice (n=3) 16 hours following immunization by subcutaneous injection with reagents as indicated. (C) Expression of co-stimulatory marker CD80 by DLN DCs collected from immunized mice above. (D-G) DCs isolated from immunized mice above were co-cultured with purified OT1 CD8+ T cells for 48 hours. Secretion of IL-12p70, IL-1α, IL-6 or TNF-α in the culture supernatant was determined by ELISA. (H) Presence of IFN-γ secreting CD8⁺ T cells was measured by ELISPOT (representative images shown to the left, and counts from 3 replicate measurements shown in the bar chart). Data presented as mean±SEM (B-H). ***P<0.001, **P<0.01, *P<0.05.

FIG. 5 demonstrates antigen-specific CTL responses induced by SNA vaccination. C57BL/6 mice (n=3) were immunized by three subcutaneous injections of SNAs or mixture of OVA1 antigen (A-D, and I) or E6 antigen (E-H and J) on days 1, 14, and 28. One week later, splenic T-cells were analyzed by flow cytometry. Percentage of CD8⁺ T-cells that were positive for CD107a (marker for cytotoxic activity) (A, E), for CD44⁺CD62L-(effector memory phenotype) (B, F), for IFN-γ (C, G). Presence of IFN-γ secreting splenic CD8⁺ T cells from immunized mice above was measured by ELISPOT 48 hours after re-stimulation ex vivo with OVA1 (D) or E6 antigen (H) (representative images shown to the left, and counts from 3 replicate measurements shown in the bar chart). Comparison of OVA1-specific (I) or E6-specific (J) cytotoxicity induced by different SNAs. Purified splenic CD8⁺ T cells from immunized mice above were co-cultured with corresponding target tumor cells at indicated ratios for 24 hours and tumor cell apoptosis was measured using Annexin V and 7-AAD staining by flow cytometry. Data presented as mean±SEM. ***P<0.001, **P<0.01, *P<0.05.

FIGS. 6A-6E depicts (a-b) activation of dendritic cells (DCs) following immunization. Mice (C57BL/6) were subcutaneously immunized with three SNA designs, as well as simple mixture of CpG and antigen (3 nmol/6 nmol) (peptide/oligonucleotide). After a 16-hour period following immunization, the expression of CD86 (a) (Biolegend, cat. 105012) and CD40 (b) (Biolegend, cat. 124626) by DCs (CD11c⁺) (Biolegend cat. 117308) was analyzed by flow cytometry. All treatment groups showed increased levels of expression of CD86 and CD40 compared to PBS group. (c-e) Absence of DC activation with complementary and anchor oligonucleotides. Purified Bone marrow-derived CD11 c⁺ DCs were treated with complementary strand (the non-CpG oligonucleotide of SNA H) or (dT)₁₀-3′-cholesterol (the non-CpG oligonucleotide of SNA A) for 2 hours at a range of concentrations (100 pM-1 uM). Upon washing the cells and incubation in fresh medium (37° C., 5% CO₂) for 24 hours, expression levels of co-stimulatory markers CD40 (c), CD80 (d), and CD86 (e) were analyzed by flow cytometry. Untreated cells served as negative controls (“Negative CTR”).

FIG. 7 shows antigen-specific T-cell proliferation induced by SNAs functionalized with C-OVA or with gp100. The eFluor 450-labeled OT1 (a) or pmel (b) splenocytes were treated ex vivo for 72 hours with SNAs formulated with C-OVA1 and C-gp100 in 10 pM concentration, respectively. Antigen specific T-cell proliferation (via dilution of eFluor 450) was compared across three different SNA structures (as well as a mixture of CpG and antigen) as indicated.

FIG. 8 shows prophylactic vaccination of LLC1-OVA tumor models with SNA structures. Mice were immunized with different SNAs (E, A and H) as well as a mixture of CpG and OVA, 19 days and 5 days before the inoculation of tumor cells (2×10⁵ LLC1-OVA cells) into the right flank of C57BL/6 mice (n=5). (a) Tumor growth for all groups treated with SNAs was significantly slower than for the untreated group or the group treated with a mixture of CpG and OVA over time. (b) Representative tumor sizes from all treated groups on day 14. There were no significant differences in tumor burden between different SNA groups. (c) The time at which tumor burden was observable (days following tumor cell inoculation) was later for treatment with SNA-H than for the other SNA treatments, and significantly later for the group treated with a mixture of CpG and OVA. (d) Kaplan-Meier survival curves of different treatment groups. SNA-H significantly increased survival of tumor-bearing mice compared to other treatments, including SNA-E and SNA-A. The survival analysis in (d) was determined by the log-rank test: ***P<0.001, **P<0.01, *P<0.05.

FIG. 9 shows that SNA structures determine the antitumor efficacy of SNA vaccination. (A) Seven days after tumor implantation, TC-1 tumor-bearing C57BL/6 mice (n=7-10) were treated with PBS, SNA-E, A, and H, or a mixture of CpG and E6 (6 nmol of CpG and 6 nmol of peptide per injection). (A) Tumor growth curves for each treatment group. (B) Survival of tumor-bearing mice shown in Kaplan-Meier curves. (C) Percentage of WBC on day 26 that are CD8⁺ T cells. (D) Percentage of WBC on day 40 that are E6-specific CD8⁺ T-cells, as determined by staining T-cells with E6 dimer. (E) Design for tumor re-challenge experiment. Memory effect and sustained rejection of tumor re-challenge in SNA H-treated mice that had rejected the initial TC-1 tumor implantation and were tumor free at least till day 72 (red line), and as a control group (black line), the growth of tumors in naïve C57BL/6 mice upon inoculation with TC-1 cells. (F) Tumor growth (F) and Kaplan-Meier survival curves (G) of LLC1-OVA-bearing C57BL/6 mice treated with SNA-E, A, or H, or mixture of CpG and OVA1. (H) Tumor growth curve of EG.7-OVA-bearing C57BL/6 mice treated with SNA-E, A, or H, or mixture of CpG and OVA1. ***P<0.001, **P<0.01, *P<0.05. Statistical significance for survival analysis in b and g was calculated by the log-rank test: ***P<0.001, **P<0.01, *P<0.05.

### DETAILED DESCRIPTION

Nanoparticle vaccines provide a way to enhance the delivery of immunostimulatory molecules to the immune system through benefits in biodistribution and co-delivery of adjuvant and antigen to immune cells¹³. Importantly, vaccine designs that use nanostructures, functionalized with both adjuvant and antigen molecules, have shown the ability to enhance the activation of antigen-presenting cells (APCs) and priming of antigen-specific cytotoxic T lymphocytes (CTLs), over that of mixtures of adjuvant and antigen molecules¹⁴. These developments underscore the need for vaccine design strategies that can effectively address multiple and specific types of immune system cells and activate corresponding pathways (e.g., antigen presentation, co-stimulatory molecular expression). Furthermore, the timing of activation and intracellular processing of vaccine components may also be crucial to creating the most active vaccines^{15,16}, and the importance of the temporal programming of dendritic cell (DC) activation by adjusting immune-cytokine injection dose and order¹⁷ has been shown. In addition, the effects of nanoparticle size and structure on the intracellular distribution of protein antigens delivered by vaccine particles¹⁸ have been investigated. Exploiting the opportunity to tune the timing and spatial control and magnitude of these pathways has the promise of optimizing the induction of anti-tumor immune responses, but requires a structural scaffold and modularity that enables the systematic study of the variables that can influence vaccine performance, while conserving other features of vaccine formulation (e.g., selection, amounts, and stoichiometric ratio of antigen and adjuvant). In some embodiments, one adjuvant is employed (i.e., only one type of adjuvant is present). Alternatively, more than one adjuvant (e.g., two, three, four, five, or more different adjuvants) are used.

SNAs are clinically used nanoparticle conjugates consisting of densely packed, highly oriented therapeutic oligonucleotides (e.g., immune-modulatory, anti-sense and siRNA gene regulatory) surrounding a nanoparticle core¹⁹⁻²². SNAs, unlike their linear cousins, possess the ability to enter cells without the need for auxiliary transfection reagents. A class of immunostimulatory SNAs (IS-SNAs) designed to activate the TLR-9 pathway and concomitantly deliver a surrogate antigen for the treatment of mouse lymphoma has been reported²³. What remained unclear in the design of SNAs as cancer vaccines however, was how differences in the chemical linkages between the nanoparticle core, oligonucleotide, and peptide can influence and provide ways to improve antigen-specific immune responses. Because IS-SNAs are well-defined nanostructures generated from chemically synthesized and purified molecular components (for example and without limitation, liposomal cores, chemically functionalized oligonucleotides, peptides), they enabled the systematic study of vaccine structure-activity-relationships, and enabled the rational and iterative design of vaccines with optimum immunostimulatory function, as disclosed herein.

The terms “polynucleotide” and “oligonucleotide” are interchangeable as used herein.

The term “associative moiety” as used herein refers to an entity that facilitates the attachment of an oligonucleotide to a SNA.

An “immune response” is a response of a cell of the immune system, such as a B cell, T cell, or monocyte, to a stimulus, such as a pathogen or antigen (e.g., formulated as an antigenic composition or a vaccine). An immune response can be a B cell response, which results in the production of specific antibodies, such as antigen specific neutralizing antibodies. An immune response can also be a T cell response, such as a CD4⁺ response or a CD8⁺ response. B cell and T cell responses are aspects of a “cellular” immune response. An immune response can also be a “humoral” immune response, which is mediated by antibodies. In some cases, the response is specific for a particular antigen (that is, an “antigen-specific response”). An immune response can be measured, for example, by ELISA-neutralization assay. Exposure of a subject to an immunogenic stimulus, such as an antigen (e.g., formulated as an antigenic composition or vaccine), elicits a primary immune response specific for the stimulus, that is, the exposure “primes” the immune response.

As used in this specification and the appended claims, the singular forms “a,” “an,” and “the” include plural reference unless the context clearly dictates otherwise.

Spherical Nucleic Acids. Spherical nucleic acids (SNAs) comprise densely functionalized and highly oriented polynucleotides on the surface of a nanoparticle which can either be organic (e.g., a liposome) inorganic (e.g., gold, silver, or platinum) or hollow (e.g., silica-based). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis(see, e.g., U.S. Patent Application Publication No. 2010/0233270, incorporated by reference herein in its entirety).

Nanoparticles are therefore provided which are functionalized to have a polynucleotide attached thereto. In general, nanoparticles contemplated include any compound or substance with a high loading capacity for a polynucleotide as described herein, including for example and without limitation, a metal, a semiconductor, a liposomal particle, insulator particle compositions, and a dendrimer (organic versus inorganic).

Thus, nanoparticles are contemplated which comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles.

Liposomal particles, for example as disclosed in International Patent Application No. PCT/US2014/068429 (incorporated by reference herein in its entirety, particularly with respect to the discussion of liposomal particles) are also contemplated by the disclosure. Hollow particles, for example as described in U.S. Patent Publication Number 2012/0282186 (incorporated by reference herein in its entirety) are also contemplated herein. Liposomal particles of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. While not meant to be limiting, the first-lipid is chosen from group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, and a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO2, Sn, SnO2, Si, SiO2, Fe, Fe+4, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992).

In practice, methods of increasing cellular uptake and inhibiting gene expression are provided using any suitable particle having oligonucleotides attached thereto that do not interfere with complex formation, i.e., hybridization to a target polynucleotide. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles particles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002, and International Application No. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Also as described in US Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in U.S. Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs (e.g., liposomal particles) is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality created by a method of the disclosure have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

Antigen. The present disclosure provides SNAs comprising an antigen. In various embodiments, the antigen is a tumor associated antigen, a tumor specific antigen, or a neo-antigen. In some embodiments, the antigen is OVA1, MSLN, P53, Ras, a melanoma related antigen (e.g., Gp100,MAGE, Tyrosinase), a HPV related antigen (e.g., E6, E7), a prostate cancer related antigen (e.g., PSA, PSMA, PAP, hTARP), an ovarian cancer related antigen (e.g., CA-125), a breast cancer related antigen (e.g., MUC-1, TEA), a hepatocellular carcinoma related antigen (e.g., AFP), a bowel cancer related antigen (e.g., CEA), human papillomavirus (HPV) E7 nuclear protein, or the SNA comprises a combination thereof. Other antigens are contemplated for use according to the compositions and methods of the disclosure; any antigen for which an immune response is desired is contemplated herein. In any of the aspects or embodiments of the disclosure, the SNA comprises a combination of two or more antigens as disclosed or taught herein.

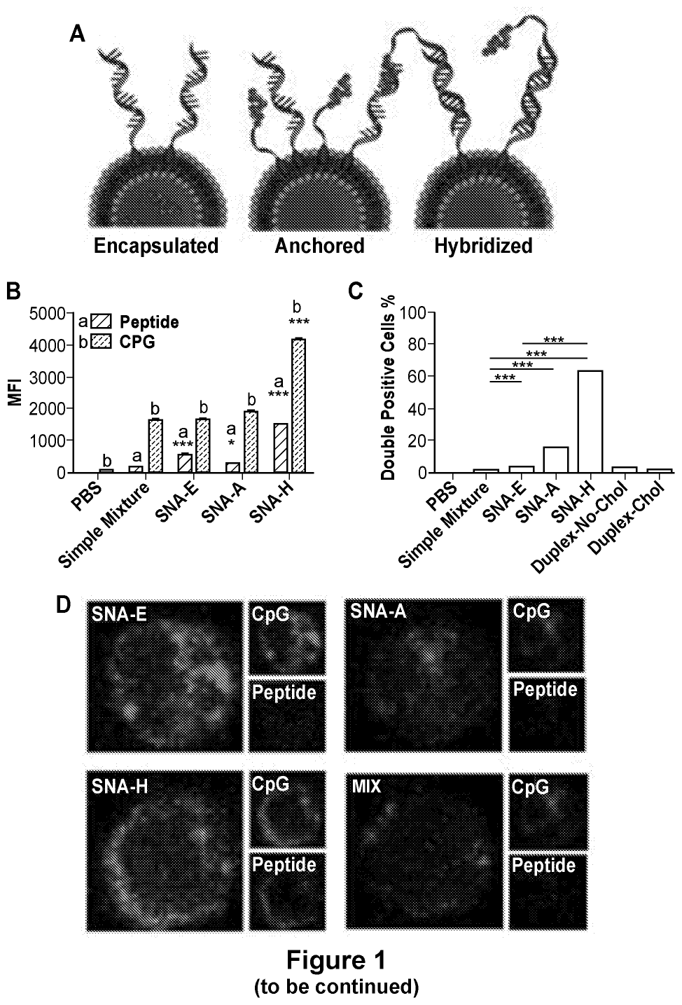

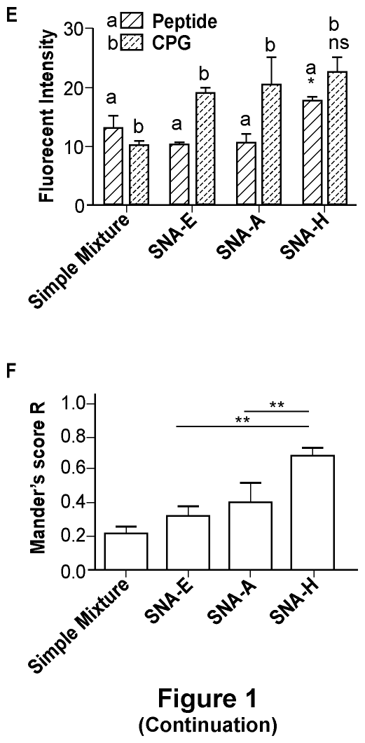

It is contemplated herein that an antigen for use in the compositions and methods of the disclosure is attached to a nucleic acid on the surface of a SNA through a linker, or attached to the surface of a SNA through a linker as disclosed herein, or both. It is contemplated that in any of the aspects of the disclosure, and as depicted in FIG. 1A, the antigen, whether attached to a nucleic acid on the surface of the SNA or attached to the surface of the SNA through a linker, is located distally with respect to the surface of the SNA. In some embodiments, an antigen is encapsulated in the SNA in addition to being surface-attached.

Linkers. The disclosure provides compositions and methods in which an antigen is associated with and/or attached to the surface of a SNA via a linker. The linker can be, in various embodiments, a cleavable linker, a non-cleavable linker, a traceless linker, and a combination thereof.

The linker links the antigen to the oligonucleotide in the disclosed SNA or links the antigen to the surface of the SNA (i.e., Antigen-LINKER-Oligonucleotide or Antigen-LINKER). The oligonucleotide can be hybridized to another oligonucleotide attached to the SNA or can be directed attached to the SNA (e.g., via attachment to an associative moiety). Some specifically contemplated linkers include carbamate alkylene, carbamate alkylenearyl disulfide linkers, amide alkylene disulfide linkers, amide alkylenearyl disulfide linkers, and amide alkylene succinimidyl linkers. In some cases, the linker comprises —NH—C(O)—O-C₂₋₅alkylene-S—S-C₂₋₇alkylene- or —NH—C(O)-C₂₋₅alkylene-S—S-C₂₋₇alkylene-. The carbon alpha to the —S—S— moiety can be branched, e.g., —CHX—S—S— or —S—S—CHY— or a combination thereof, where X and Y are independently Me, Et, or iPr. The carbon alpha to the antigen can be branched, e.g., —CHX-C₂₋₄alkylene-S—S-, where X is Me, Et, or iPr. In some cases, the linker is —NH—C(O)—O—CH₂—Ar—S—S -C₂₋₇alkylene-, and Ar is a meta- or para-substituted phenyl. In some cases, the linker is —NH—C(O)-C₂₋₄alkylene-N-succinimidyl-S-C₂₋₆alkylene-.

Additional linkers include an SH linker, SM linker, SE linker, and SI linker. The disclosure contemplates multiple points of attachment available for modulating antigen release (e.g., disulfide cleavage, linker cyclization, and dehybridization), and the kinetics of antigen release at each attachment point can be controlled. For example, steric bulk about the disulfide can decrease the rate of the S_{N}2 reaction; increased length of an alkyl spacer or steric bulk attached to the alkyl spacer can affect the rate of ring closure; and mismatched nucleotide sequences lower the melting temperature (Tₘ), while locked nucleic acids increase the Tₘ.

Polynucleotides. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynylcytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and International Patent Publication No. WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1 H-pyrimido[5,4-b][1,4]benzoxazin-2(3H)-one), phenothiazine cytidine (1 H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox- azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as in embodiments relating to hybridization to a target polynucleotide, the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide. In some embodiments, the disclosure contemplates that a polynucleotide attached to a nanoparticle is RNA. The RNA can be either single-stranded or double-stranded, so long as it is able to hybridize to a target polynucleotide.

In some aspects, multiple polynucleotides are functionalized to a nanoparticle. In various aspects, the multiple polynucleotides each have the same sequence, while in other aspects one or more polynucleotides have a different sequence. In some embodiments, the one or more polynucleotides having a different sequence target more than one gene product. In further aspects, multiple polynucleotides are arranged in tandem and are separated by a spacer. Spacers are described in more detail herein below.

Polynucleotide attachment to a nanoparticle. Polynucleotides contemplated for use in the methods include those bound to the nanoparticle through any means (e.g., covalent or non-covalent attachment). Regardless of the means by which the polynucleotide is attached to the nanoparticle, attachment in various aspects is effected through a 5′ linkage, a 3′ linkage, some type of internal linkage, or any combination of these attachments. In some embodiments, the polynucleotide is covalently attached to a nanoparticle. In further embodiments, the polynucleotide is non-covalently attached to a nanoparticle. An oligonucleotide of the disclosure comprises, in various embodiments, an associative moiety selected from the group consisting of a tocopherol, a cholesterol moiety, DOPE-butamide-phenylmaleimido, and lyso-phosphoethanolamine-butamide-pneylmaleimido. See also U.S. Patent Application Publication No. 2016/0310425, incorporated by reference herein in its entirety.

Methods of attachment are known to those of ordinary skill in the art and are described in U.S. Publication No. 2009/0209629, which is incorporated by reference herein in its entirety. Methods of attaching RNA to a nanoparticle are generally described in International Patent Application No. PCT/US2009/65822, which is incorporated by reference herein in its entirety. Methods of associating polynucleotides with a liposomal particle are described in International Patent Application No. PCT/US2014/068429, which is incorporated by reference herein in its entirety.

Spacers. In certain aspects, functionalized nanoparticles are contemplated which include those wherein an oligonucleotide is attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to the target polynucleotide. In certain aspects, the bases of the polynucleotide spacer are all adenylic acids, all thymidylic acids, all cytidylic acids, all guanylic acids, all uridylic acids, or all some other modified base.

Nanoparticle surface density. A surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of polynucleotide on the surface of the SNA is measured by the number of polynucleotides on the surface of a SNA. With respect to the surface density of polynucleotides on the surface of a SNA of the disclosure, it is contemplated that a SNA as described herein comprises from about 1 to about 100 oligonucleotides on its surface. In various embodiments, a SNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface. In further embodiments, a SNA comprises at least about 5, 10, 20, 30, 40, 45, 50, 55, 60, 65, 70, 75, 80, 85, 90, 95, 100, 110, 120, 130, 140, 150, 160, 170, 180, 190, or 200 polynucleotides on its surface.

### METHODS

The disclosure generally provides methods for testing and/or selecting a SNA to determine the kinetics of antigen presentation and generation of a costimulatory signal in an antigen-presenting (e.g., dendritic) cell. It will be understood that while dendritic cells are exemplified and discussed herein throughout, any antigen-presenting cell is contemplated for use according to the methods described herein. Dendritic cells, macrophages, and B cells are the principal antigen-presenting cells for T cells, whereas follicular dendritic cells are the main antigen-presenting cells for B cells. Lymphocytes are also contemplated by the disclosure. The immune system contains three types of antigen-presenting cells, i.e., macrophages, dendritic cells, and B cells. The use of any antigen-presenting cell is contemplated by the disclosure.

Accordingly, in some aspects, the disclosure provides a method comprising treating a population dendritic cells (DCs) with a spherical nucleic acid (SNA) comprising a nanoparticle, an antigen, and an adjuvant; and determining a time at which the population of DCs presents a maximal signal that is indicative of antigen presentation by the DCs and a time at which the population of DCs presents a maximal co-stimulatory signal due to the adjuvant.

In further aspects, the disclosure provides a method of selecting a spherical nucleic acid (SNA) for increased ability to activate dendritic cells (DCs), comprising: generating a first SNA comprising a nanoparticle, an antigen, and an adjuvant and a second SNA comprising nanoparticle, an antigen, and an adjuvant; treating a first population of dendritic cells (DCs) with the first SNA and treating a second population of DCs with the second SNA; determining a time at which the first population of DCs presents a maximal signal that is indicative of antigen presentation and a time at which the first population of DCs presents a maximal co-stimulatory signal due to the adjuvant; determining a time at which the second population of DCs presents a maximal signal that is indicative of antigen presentation and a time at which the second population of DCs presents a maximal co-stimulatory signal due to the adjuvant; and selecting as the SNA for which time to achieve maximal signal for antigen presentation is the same as or about the same as time to achieve maximal co-stimulatory signal.

In any of the aspects described therein, one adjuvant may be employed (i.e., only one type of adjuvant is present), or more than one adjuvant (e.g., two, three, four, five, or more different adjuvants) may be employed. In any of the aspects described herein, one antigen may be employed (i.e., only one type of antigen is present), or more than one antigen (e.g., two, three, four, five, or more different antigens) may be employed.

Various parameters of the SNA structure may be varied in designing an immunotherapeutic agent according to the disclosure. For example and without limitation, one can vary the core material of the SNA (e.g., liposomal, metallic) the density and species of oligonucleotides on the surface of the SNA, the density of antigen on the surface of the SNA or encapsulated within the SNA, the type of attachment used to attach one or more antigens to the surface of the SNA (e.g., attached through an oligonucleotide that is attached to the surface of the SNA, or attached directly to the surface of the SNA through a linker), the identity of the linker used for antigen attachment, or a combination of the foregoing parameters. Each of the foregoing parameters is discussed in further detail herein. By varying the structure of the SNA and performing a method as described and exemplified herein, one can maximize the therapeutic efficacy of the SNA.

### Uses of SNAs in Gene Regulation/Therapy

In addition to serving a role in providing an oligonucleotide (e.g., an immunostimulatory oligonucleotide) and an antigen to a cell, it is also contemplated that in some embodiments, a SNA of the disclosure possesses the ability to regulate gene expression. Thus, in some embodiments, a SNA of the disclosure comprises an antigen that is associated with a SNA through a linker, an oligonucleotide (e.g., an immunostimulatory oligonucleotide), and an additional oligonucleotide having gene regulatory activity (e.g., inhibition of target gene expression or target cell recognition). Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an oligonucleotide which is 100% complementary to the target polynucleotide, i.e., a perfect match, while in other aspects, the oligonucleotide is about or at least (meaning greater than or equal to) about 95% complementary to the polynucleotide over the length of the oligonucleotide, about or at least about 90%, about or at least about 85%, about or at least about 80%, about or at least about 75%, about or at least about 70%, about or at least about 65%, about or at least about 60%, about or at least about 55%, about or at least about 50%, about or at least about 45%, about or at least about 40%, about or at least about 35%, about or at least about 30%, about or at least about 25%, about or at least about 20% complementary to the polynucleotide over the length of the oligonucleotide to the extent that the oligonucleotide is able to achieve the desired degree of inhibition of a target gene product. Moreover, an oligonucleotide may hybridize over one or more segments such that intervening or adjacent segments are not involved in the hybridization event (e.g., a loop structure or hairpin structure). The percent complementarity is determined over the length of the oligonucleotide. For example, given an inhibitory oligonucleotide in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Accordingly, methods of utilizing a SNA of the disclosure in gene regulation therapy are provided. This method comprises the step of hybridizing a polynucleotide encoding the gene with one or more oligonucleotides complementary to all or a portion of the polynucleotide, the oligonucleotide being the additional oligonucleotide of a composition as described herein, wherein hybridizing between the polynucleotide and the additional oligonucleotide occurs over a length of the polynucleotide with a degree of complementarity sufficient to inhibit expression of the gene product. The inhibition of gene expression may occur in vivo or in vitro.

The oligonucleotide utilized in the methods of the disclosure is either RNA or DNA. The RNA can be an inhibitory RNA (RNAi) that performs a regulatory function, and in various embodiments is selected from the group consisting of a small inhibitory RNA (siRNA), an RNA that forms a triplex with double stranded DNA, and a ribozyme. Alternatively, the RNA is microRNA that performs a regulatory function. The DNA is, in some embodiments, an antisense-DNA.

### Use of SNAs in Immune Regulation

Toll-like receptors (TLRs) are a class of proteins, expressed in sentinel cells, that plays a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotide are located inside special intracellular compartments, called endosomes. The mechanism of modulation of TLR 4, TLR 8 and TLR9 receptors is based on DNA-protein interactions.

Synthetic immunostimulatory oligonucleotides that contain CpG motifs that are similar to those found in bacterial DNA stimulate a similar response of the TLR receptors. Therefore immunomodulatory oligonucleotides have various potential therapeutic uses, including treatment of immune deficiency and cancer.

In some embodiments, the disclosure provides a method of up-regulating activity of a TLR comprising contacting a cell having the TLR with a SNA of the disclosure. In further embodiments, the cell is an antigen presenting cell (APC). In some embodiments, the APC is a dendritic cell, while in further embodiments the cell is a leukocyte. The leukocyte, in still further embodiments, is a phagocyte, an innate lymphoid cell, a mast cell, an eosinophil, a basophil, a natural killer (NK) cell, a T cell, or a B cell. The phagocyte, in some embodiments, is a macrophage, a neutrophil, or a dendritic cell.

Down regulation of the immune system would involve knocking down the gene responsible for the expression of the Toll-like receptor. This antisense approach involves use of SNAs conjugated to specific antisense oligonucleotide sequences to knock down the expression of any toll-like protein.

Accordingly, methods of utilizing SNAs for modulating toll-like receptors are disclosed. The method either up-regulates or down-regulates the Toll-like-receptor through the use of a TLR agonist or a TLR antagonist, respectively. The method comprises contacting a cell having a toll-like receptor with a SNA of the disclosure. The toll-like receptors modulated include toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

Compositions. The disclosure includes compositions that comprise a pharmaceutically acceptable carrier and a spherical nucleic acid (SNA) of the disclosure, wherein the SNA comprises a nanoparticle, an oligonucleotide on the surface of the nanoparticle (which, in any of the aspects or embodiments of the disclosure, serves as an adjuvant), and an antigen that is associated with the surface of the SNA via a linker. In some embodiments, the composition is an antigenic composition. The term “carrier” refers to a vehicle within which the SNA is administered to a mammalian subject. The term carrier encompasses diluents, excipients, an additional adjuvant and a combination thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

Additional adjuvants (i.e., adjuvants in addition to the adjuvant that is associated with an SNA of the disclosure) include but are not limited to emulsions, microparticles, immune stimulating complexes (iscoms), LPS, CpG, or MPL.

Methods of inducing an immune response. The disclosure includes methods for eliciting an immune response in a subject in need thereof, comprising administering to the subject an effective amount of a composition or vaccine of the disclosure. In some embodiments, the vaccine is a cancer vaccine. In further embodiments, the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.

The immune response raised by the methods of the present disclosure generally includes an innate and adaptive immune response, preferably an antigen presenting cell response and/or CD8+ and/or CD4+ T-cell response and/or antibody secretion (e.g., a B-cell response). The immune response generated by a composition as disclosed herein is directed against, and preferably ameliorates and/or neutralizes and/or reduces the tumor burden of cancer. Methods for assessing immune responses after administration of a composition of the disclosure (immunization or vaccination) are known in the art and/or described herein. Antigenic compositions can be administered in a number of suitable ways, such as intramuscular injection, subcutaneous injection, intradermal administration and mucosal administration such as oral or intranasal. Additional modes of administration include but are not limited to intranasal administration, and oral administration.

Antigenic compositions may be used to treat both children and adults. Thus a subject may be less than 1 year old, 1-5 years old, 5-15 years old, 15-55 years old, or at least 55 years old.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Administration of more than one dose (typically two doses) is particularly useful in immunologically naive subjects or subjects of a hyporesponsive population (e.g., diabetics, or subjects with chronic kidney disease). Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). Preferably multiple doses are administered from one, two, three, four or five months apart. Antigenic compositions of the present disclosure may be administered to patients at substantially the same time as (e.g., during the same medical consultation or visit to a healthcare professional) other vaccines.

Articles of Manufacture and Kits. The disclosure additionally includes articles of manufacture and kits comprising a composition described herein. In some embodiments, the kits further comprise instructions for measuring antigen-specific antibodies. In some embodiments, the antibodies are present in serum from a blood sample of a subject immunized with a composition comprising a SNA of the disclosure.

As used herein, the term “instructions” refers to directions for using reagents contained in the kit for measuring antibody titer. In some embodiments, the instructions further comprise the statement of intended use required by the U.S. Food and Drug Administration (FDA) in labeling in vitro diagnostic products.

The following examples illustrate various embodiments contemplated by the present disclosure. The examples are exemplary in nature and are in no way intended to be limiting.

### EXAMPLES

The Examples describe a comparison of three SNA structures clearly differentiated in the chemistry of antigen incorporation. The ability of these structures to induce antigen-specific immune responses in several mouse models of cancer was investigated. These designs were chosen to evaluate the importance of SNA structure on their ability to: 1) co-deliver antigen and adjuvant to individual APCs (and not just populations of APCs); 2) control the kinetics of release of adjuvant and antigen from the SNA, and timing of antigen presentation and DC activation; 3) lead to intracellular processing of peptide antigen for effective presentation by the MHC-I pathway (cross-presentation). These functions are essential for generating antigen-specific immune response and performing as vaccines. Orchestrating the co-delivery and timing of immunostimulatory pathways may lead to successful induction of antigen-specific CTLs, while poor coordination of these events (e.g., induction of co-stimulatory markers but not of antigen presentation, or of antigen presentation without co-stimulatory markers) could lead to T-cell fatigue or anergy.

Three SNA structures that are compositionally nearly identical but structurally different markedly varied in their abilities to cross-prime antigen-specific CD⁸⁺ T-cells and raise subsequent anti-tumor immune responses. Importantly, the most effective structure was the one that exhibited synchronization of maximum antigen presentation and costimulatory marker expression. In the HPV-associated TC-1 model, vaccination with this structure improved overall survival, induced the complete elimination of tumors from 30% of the mice, and conferred curative protection from tumor re-challenges, consistent with immunological memory not otherwise achievable. The antitumor effect of SNA vaccination was dependent on the method of antigen incorporation within the SNA structure, underscoring the modularity of this novel class of nanostructures and the potential for the deliberate design of new vaccines, thereby defining a rational cancer vaccinology.

In designing the three SNAs, the aim was to conserve composition (i.e., TLR9-agonist oligonucleotide, peptide antigen, nanoparticle core) but to vary the position and conjugation chemistry of the peptide antigen. Each of the three SNA structures consisted of a unilamellar liposome core (40-45-nm in diameter, DOPC) that both presented and oriented TLR9 agonist oligonucleotides (3′-cholesterol-functionalized, “1826” CpG sequence specific for the activation of murine TLR9) at the surface. The three SNA architectures (E, A, and H) examined varied in the position and conjugation chemistry of the peptide antigen in the following ways: 1) soluble antigen encapsulated within the liposome core (“encapsulated” model, E); 2) antigen located at the surfaces of SNAs, by chemical conjugation to oligonucleotides (functionalized at the 3′-terminus with cholesterol groups) adsorbed to the liposome surface (“anchored” model, A); 3) antigen located at the surfaces of SNAs, by chemical conjugation of the antigen to oligonucleotides hybridized to CpG oligonucleotides adsorbed to the liposome surface (“hybridized” model, H). For antigens chemically conjugated to oligonucleotides, we used a biochemically labile linker for the traceless release of antigen was used, as previously described²⁴. For each of the three SNA structures, three different peptide antigens were used to evaluate immune responses in vitro and in vivo: OVA1 (C-SIINFEKL(SEQ ID NO: 1)), melanoma derived antigen gp100 (C-KVPRNQDWL (SEQ ID NO: 2)), and HPV-16 oncoprotein E6 antigen (VYDFAFRDLC (SEQ ID NO: 3)). The influence of these structural variations on the uptake, co-delivery of CpG and antigen, intracellular trafficking and retention of antigen, kinetics of activation and antigen presentation, induction of antigen-specific CD⁸⁺ T-cell responses, and ultimately in vivo antitumor efficacy, was evaluated. These activities were also compared to those of “unformulated” vaccines: mixtures of soluble TLR9-agonist and peptide antigen, without any chemical conjugation.

### Example 1

### Design and Synthesis of SNAs With Variation in Antigen Incorporation

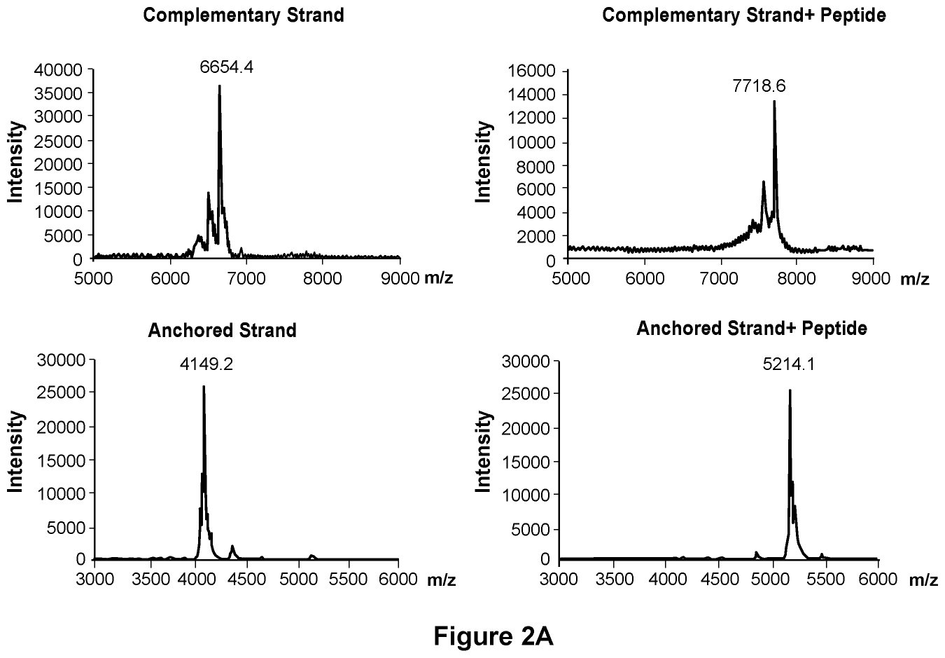

The approach to generating well-differentiated SNA structures E, A, and H took advantage of the modular nature and chemical synthesis of SNAs (FIG. 1A). Each of the molecular components of these SNAs was synthesized and purified (chemically functionalized oligonucleotides, peptides, liposomes), and incorporated into the liposomal SNA structure through the initial formation of liposomes, followed by the adsorption of the adjuvant to their surfaces via hydrophobic anchoring groups (cholesterol). For SNA E, antigen was loaded into the core during the liposome formation process. For SNA A, a peptide-oligonucleotide-3′-cholesterol conjugate was co-adsorbed to liposomes along with 3′-cholesterol-functionalized CpG. For SNA H, a peptide-oligonucleotide conjugate, with a nucleotide sequence complementary to CpG, was hybridized with CpG oligonucleotides prior to adsorption to liposomes. Details for the synthetic procedures and the characterization of the physical properties and chemical composition of the SNAs are below (FIG. 2a-e). To compare SNAs that differ in structure, but not in composition, E, A, and H SNAs were prepared that were similar in the stoichiometry of CpG and antigen to liposome (75 molecules of each per liposomal structure with an average diameter of 55-60 nm, including the oligonucleotide shell) (FIG. 2f). SNAs E, A, and H were synthesized with different antigens (OVA-1, gp100, E6), and subsequently their immunostimulatory properties were compared and their performance as therapeutic vaccines explored in clinically relevant mouse tumor models.

### Synthesis of SNAs

The synthesis of SNAs involves the three steps of 1) oligonucleotide synthesis; 2) liposome formation; 3) adsorption of oligonucleotides to liposomes and purification.

### Oligonucleotide (DNA) Synthesis

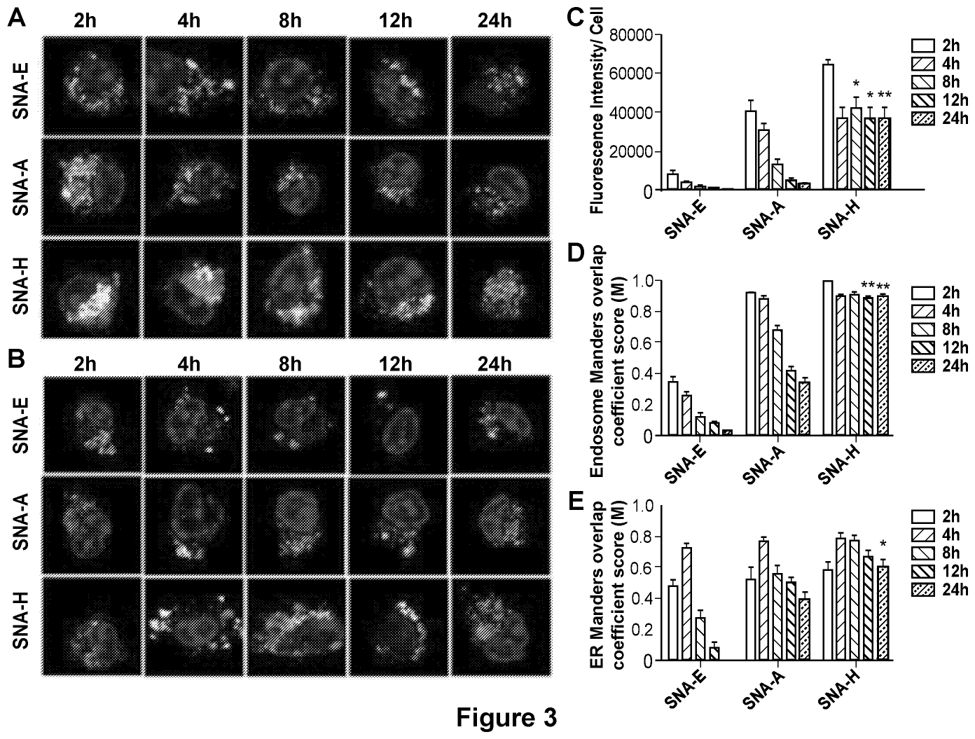

Cholesterol terminated CpG DNA, DNA with complementary sequence, and DNA for anchoring chemically conjugated peptides (sequences shown below in Table 1) were synthesized using automated solid-support phosphoramidite synthesis on an Expedite 8909 Nucleotide Synthesis System or MM48 Synthesizer, Bioautomation, Plano, Tex., USA, with DCI as an activator. All oligonucleotides were synthesized with phosphorothioate backbones (PS) through the use of 3-((Dimethylamino-methylidene)amino)-3H-1,2,4-dithiazole-3-thione as sulfurizing agent. The C6-thiolated phosphoramidite (for SNA A) was coupled to the (dT)₁₀, cholesterol-terminated DNA oligonucleotides using an extended coupling time of 15 minutes. After the completion of solid phase synthesis, oligonucleotide strands were cleaved from the solid support by overnight treatment with aqueous ammonium hydroxide (28-30 wt % aqueous solution, Aldrich Chemicals, Milwaukee, Wis., USA), after which the excess ammonia was removed by evaporation. Oligonucleotides were purified using a Microsorb C4 or C18 column on a high pressure liquid chromatography system (Varian ProStar Model 210, Varian, Inc., Palo Alto, Calif., USA) using a gradient of aqueous TEAA (triethylammonium acetate) and acetonitrile (10% v/v to 100% acetonitrile over 30 minutes). The product-containing fractions were collected and concentrated by lyophilization. The oligonucleotides were re-suspended in ultrapure deionized water, and analyzed by MALDI-TOF and denaturing polyacrylamide gel electrophoresis. The conjugation of peptides to —SH functionalized oligonucleotides was accomplished by disulfide exchange reactions with cysteine-containing peptides (C-OVA1, C-gp100, E6) activated by 4,4′-dithiodipyridine and purified by denaturing PAGE, or by disulfide exchange reactions with OVA1 functionalized with (4-nitrophenyl 2-(2-pyridyldithio)ethyl carbonate (NDEC) “traceless” linker and purified with denaturing PAGE [Skakuj, K. et al. Conjugation Chemistry-Dependent T-Cell Activation with Spherical Nucleic Acids. Journal of the American Chemical Society 140, 1227-1230 (2018)]. Analysis of the synthetic oligonucleotides and C-OVA1-conjugated oligonucleotides by MALDI-TOF-MS is shown in FIG. 2a. The preparation of duplex DNA (for SNA H only) is shown in FIG. 2b. Data collected for evaluating co-delivery and imaging used TMR-labeled OVA1 that was either encapsulated in liposome core (SNA-E), or conjugated to anchored strand (SNA-A) or complementary strand (SNA-H) with the NDEC linker (FIG. 1). Data collected for evaluating immune responses (FIGS. 3-5) used C-OVA1, Cgp100, and E6 (V10C) as antigen.

TABLE 1
Sequences of synthetic oligonucleotides.

| Strand Name | Sequence | SEQ ID NO: |
|---|---|---|
| CpG-3′-cholesterol | 5′-TCC ATG ACG TTC CTG ACG TT (Sp18)₂ Cholesterol-3′(PS) | 4 |
| CpG used in simple | 5′-TCC ATG ACG TTC CTG ACG TT (Sp18)₂ TT-3′ (PS) | 5 |
| mixtures with antigen | | |
| Cy5-CpG-3′-cholesterol | 5′-TCC ATG ACG TTC CTG ACG TT-Cy5-(Sp18)₂ Cholesterol-3′ (PS) | 6 |
| Cy5-CpG used in simple | 5′-TCC ATG ACG TTC CTG ACG TT-Cy5-(Sp18)₂ TT-3′ (PS) | 7 |
| mixtures with TMR-OVA1 | | |
| Complementary | 5′-AAC GTC AGG AAC GTC ATG GA-SH-3′ (PS) | 8 |
| Strand (used for SNA H) | | |
| (dT)₁₀-3′-cholesterol | 5′-SH-TTT TTT TTT T Cholesterol-3′ (PS) | 9 |

### Peptide Antigens

All peptide antigens used in this study were obtained by custom synthesis by Genscript at >95% purity and used without further purification. Table 2 contains the amino acid sequences of the peptides.

TABLE 2
Sequences of peptide antigen

| Peptide | Peptide Sequences | SEQ ID NO: |
|---|---|---|
| OVA1 | SIINFEKL | 10 |
| C-OVA1 | CSIINFEKL | 1 |
| TMR-OVA1 | TMR-α-NH-SIINFEKL | 11 |
| C-gp100 | CKVPRNQDWL | 12 |
| E6 (V10C) | VYDFAFRDLC | 13 |

### Liposome Synthesis

Liposome cores for SNAs were prepared using a modification of a published protocol [Radovic-Moreno, A. F. et al. Immunomodulatory spherical nucleic acids. Proceedings of the National Academy of Sciences 112, 3892-3897 (2015); Banga, R. J., Chernyak, N., Narayan, S. P., Nguyen, S. T. & Mirkin, C. A. Liposomal Spherical Nucleic Acids. Journal of the American Chemical Society 136, 9866-9869 (2014).]. Chloroform solutions of di-oleoyl phosphatidylcholine (DOPC) (2 mL, 25 mg/mL concentration) were added to glass vials, and the solvent was removed by evaporation with a stream of nitrogen; residual chloroform was removed by vacuum for greater than 12 hours. The resulting film of DOPC was hydrated with solutions of phosphate buffered (PBS) (pH=7.4) for SNA-A and SNA-H), or solutions containing peptides for SNA-E (2 mgs/mL). Following vortexing, the resulting suspensions were treated with 10 freeze-thaw cycles, and then extruded through a series of polycarbonate membranes (200 nm, 100 nm, 50 nm pore sizes; Avanti Polar Lipids, Inc.). The extruded DOPC liposomes were then analyzed by dynamic light scattering (DLS; FIG. 2c) and Cryo-EM (FIG. 2e). Unencapsulated peptide in the preparation of SNA-E was removed by dialysis or tangential flow filtration (100-kDa membranes from Spectrum Chromatography). The final DOPC and peptide concentrations in extruded samples were determined by spectroscopic analysis with commercially available reagent kits for DOPC or for peptides using standard curves generated for C-OVA, Cgp100, and E6 (Sigma, MAK049 USA; ThermoFisher, Cat:23290). Average values of the stoichiometry of peptide encapsulation for SNA E were 15-20, approximately 75, and approximately 75 for OVA1 and C-OVA1, gp100, and E6, respectively.

### SNA Assembly

The general procedure for the synthesis of SNAs involves the mixing of DNA or DNA duplexes with liposomes in an approximate 75:1 ratio (mol/mol) and dilution with PBS to form solutions with a concentration of 50 μM by DNA or DNA duplex; this DNA:liposome stoichiometry uses the assumption of 18,132 DOPC molecules per 50-nm, unilamellar liposome. Solutions were shaken 400 rpm at 37° C. overnight and then used without further purification. The characterization of SNAs by zeta potential is shown in FIG. 2d and by cryo-electron microscopy is shown in FIG. 2e. The analysis of SNAs by gel electrophoresis (1% agarose, tris-borate-EDTA), followed by staining with SYBR Green II is provided in FIG. 2f.

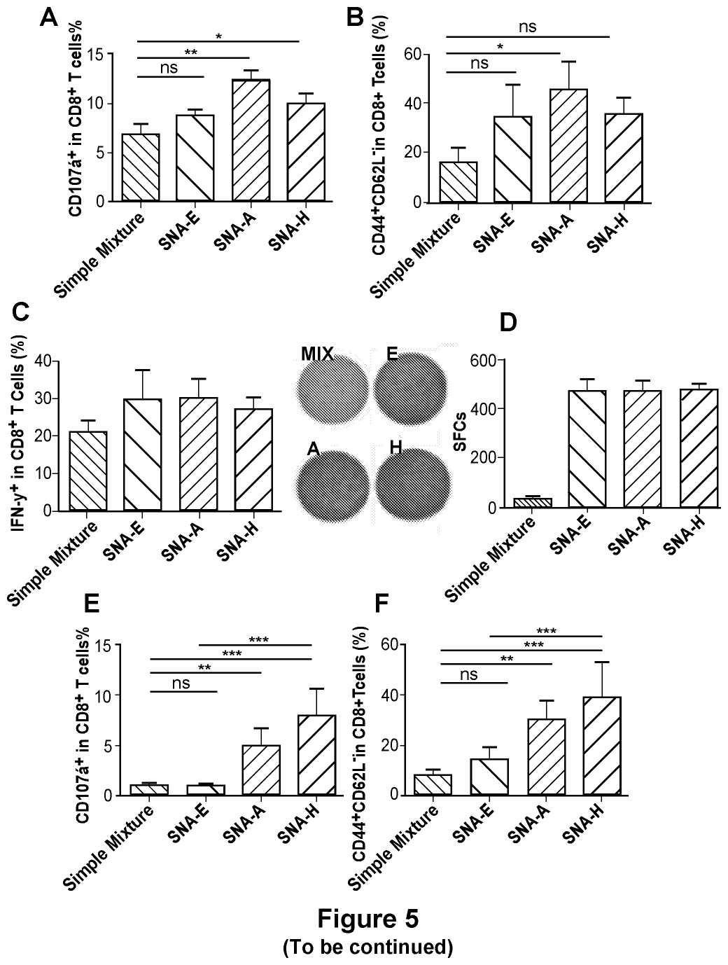

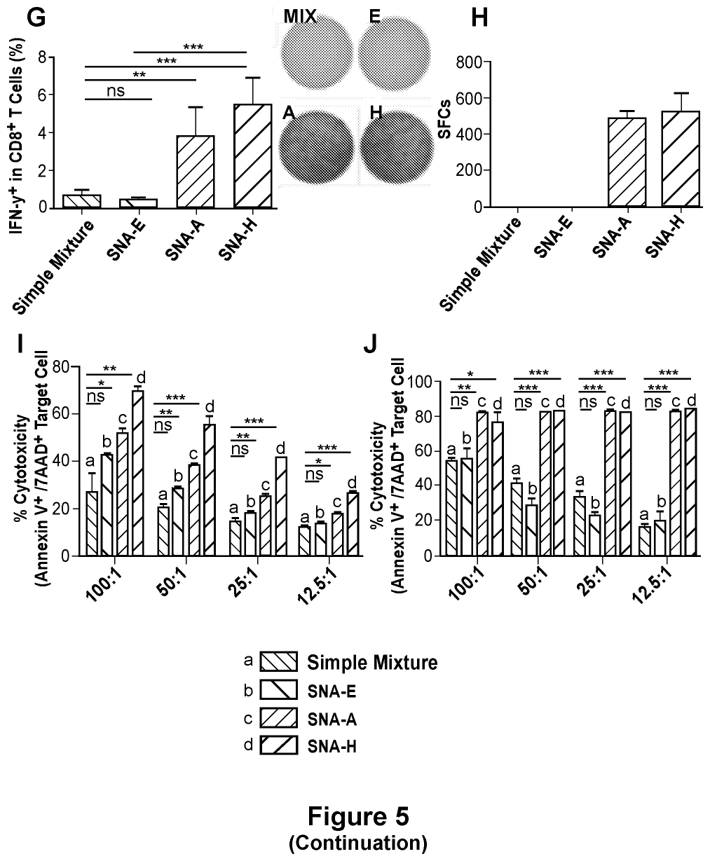

For the studies of co-delivery of TMR-OVA and Cy5 CpG (FIG. 1) and for anti-tumor efficacy for tumor models with LLC-OVA or TC-1 cells (FIG. 5), the ratio of peptide antigen to CpG was 1:1. For SNA E, liposomes with encapsulated peptide were used; the number of CpG-3′-cholesterol oligonucleotides added per liposome was the same as the stoichiometry of encapsulated peptide per liposome (15-20 for OVA1 and C-OVA1, and 75 for gp100 and E6). For SNA A, the 75:1 oligonucleotide:liposome ratio was attained by the addition of 37.5 peptide-conjugated (dT)10-3′-cholesterol and 37.5 CpG-3′-cholesterol oligonucleotides per liposome. For SNA H, 75 duplex DNA oligonucleotides were added per liposome.

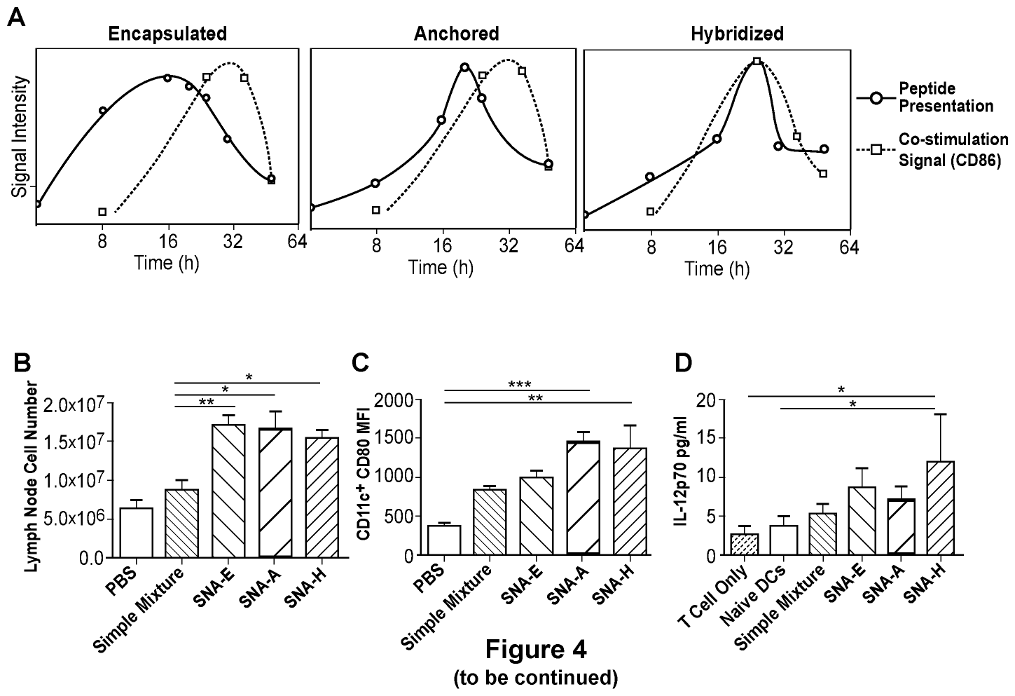

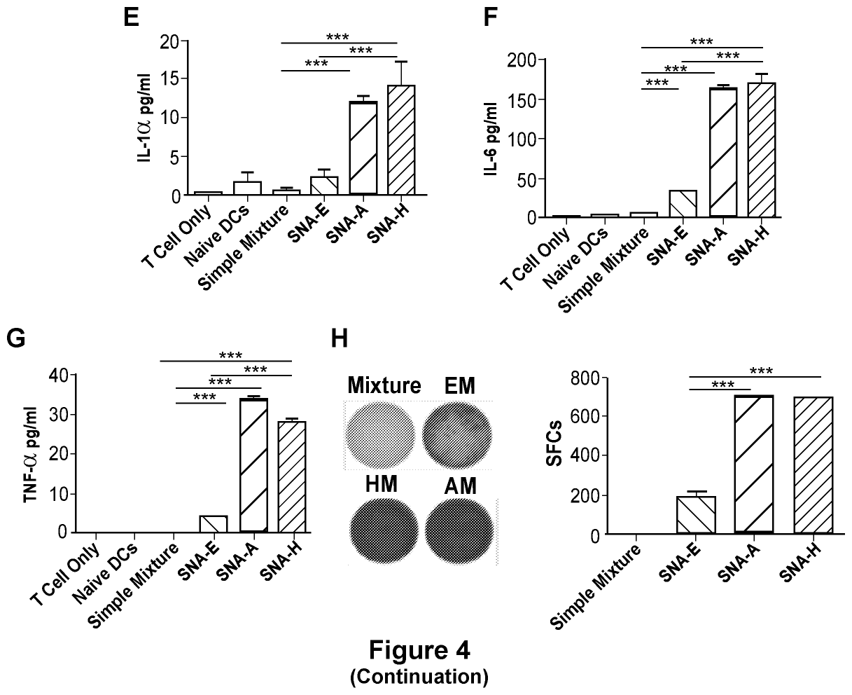

For the studies of DC activation (FIG. 3) and T-cell activation (FIG. 4), the ratio of peptide antigen to CpG was 1:2. For SNA E, liposomes with encapsulated peptide were used; the number of CpG-3′-cholesterol oligonucleotides added per liposome (40 for OVA1, 75 for gp100 and E6) was twice the stoichiometry of encapsulated peptide per liposome (20 for OVA1 and approximately 40 for gp100 and E6). For SNA A, the 75:1 oligonucleotide:liposome ratio was attained by the addition of 25 peptide-conjugated (dT)10-3′-cholesterol and 50 CpG-3′-cholesterol oligonucleotides per liposome. For SNA H, 37.5 duplex DNA oligonucleotides (with conjugated peptide) and 37.5 CpG-3′-cholesterol were added per liposome.

### Evaluating the Ability of Different SNA Structures to Co-Deliver Immunostimulatory Oligonucleotides and Peptide Antigens to DCs

The ability of E, A, and H SNA structures to enter DCs and deliver both CpG oligonucleotides and peptide antigens to individual DCs was compared. The delivery of both types of molecules, and the induction of signaling for the parallel pathways of antigen presentation and co-stimulatory marker expression, are essential steps for activating APCs and further priming antigen-specific T-cells. Upon treatment of bone marrow-derived DCs (BMDCs) with each SNA structure functionalized with CpG (labeled with Cy5) and OVA1 antigen (labeled with TMR) and analysis of cellular uptake, significant advantages for SNA H in the uptake of both CpG and antigen was found (FIG. 1 B). To investigate these effects in vivo, mice were injected subcutaneously with the same set of SNAs. Extraction of the draining lymph node (DLN) after 2 hours and analysis of the CD11c⁺ DCs by flow cytometry showed a wide range in the fraction of cells containing high levels of both CpG and OVA1. The fraction of DCs with high levels of uptake for both CpG and OVA1 depended on SNA structure and followed the order of E<A<H. Indeed, SNA H remarkably led to greater than 60% of a DC population showing co-delivered adjuvant and antigen, far greater than that for SNAs E and A (FIG. 1C). In contrast, for mixtures of CpG and OVA1 (no coupling between the components), the fraction of DCs showing co-delivery was negligible (less than 1.5%). The comparison of results for SNA H and dsDNA conjugated to OVA1 that is not formulated into SNA structure (less than 2% co-delivery) established the critical influence of SNA structure in achieving high levels of co-delivered oligonucleotide and peptide. These data showed that the dependence of the co-delivery of CpG and antigen on SNA structure, and the superiority of SNA H, are amplified in vivo. The structural features of SNA H that drive the enhancement of co-delivery are: 1) the linkage of antigen to CpG by chemical conjugation and nucleic acid hybridization, and 2) the enhancement of cellular uptake of oligonucleotides by the SNA architecture. SNA H is not susceptible to erosion in co-delivery through the mechanisms likely responsible for separation of antigen and CpG in SNAs E and A (i.e., leakage of peptide through liposome membranes, and desorption of antigen-functionalized oligonucleotides from liposomes).

The co-delivery of adjuvant and antigen molecules by SNAs was analyzed by imaging (via confocal microscopy) the DCs extracted from mice immunized by SNAs with Cy5-labeled CpG and TMR-labeled OVA. The images showed comparable levels of CpG delivered by each SNA structure, but higher levels of OVA1 co-delivered by SNA H than those by SNAs A and E (FIGS. 1D, 1E). Manders coefficient values (FIG. 1F) showed a decreasing r score for SNAs H (r=0.68), A (r=0.40), and E (r=0.32), indicating that the highest levels of subcellular co-localization of CpG and OVA1 are accomplished by SNA H, at an early time point (4 hours after vaccination) when intracellular processing of antigen is at an early stage.

### The Trafficking of Peptide Antigens Within DCs, Delivered by Different SNA Structures

The uptake, trafficking, and retention of peptide antigens delivered by SNA-E, A, and H was compared. Upon treatment of BMDCs with SNA structures formulated with OVA1 labeled with Cy5 for 2 hours, the cells were washed and incubated in fresh medium and monitored by confocal fluorescence microscopy over a further 24 hour period. Presence of OVA1 in late endosomes and endoplasmic reticulum (ER) was determined by co-localization of Cy5 (red) and fluorescent markers (green) for the late endosomes and the ER, respectively, in confocal microscope images (FIGS. 3A and 3B). Clear trends were found that differentiate the SNA structures in the uptake of OVA1 (at the earliest time points of 2 hours and 4 hours), and in the retention of OVA1 at the late time points. The order in overall delivery of OVA1 is H >A >E at the early time point of 2 hours. At 24 hours, only SNA H enabled substantial retention of peptide within the cells (57% of the maximum levels observed at 2 hours). Both SNA-E and SNA-A however showed a rapid decline in the presence of peptide (less than 8% of maximum levels observed at 2 hours) (FIG. 3C). The subsequent analysis of subcellular distribution of OVA1 indicated that this effect was driven by the sustained retention of OVA1 delivered by SNA H in the endosome (FIG. 3D) and ER (FIG. 3E), the site of MHC-1 peptide loading, through the 24 hour period following SNA treatment. The higher uptake of peptide antigen delivered by SNA H, followed by retention at substantial levels of these peptides in the endocytic pathway and ER for a 24-hour period, is dependent on the structure of SNA H, and provides a major advantage in generating longer windows of time for efficient cross-priming of antigen-specific T-cells by DCs.

### Activation of DCs and Cross-Priming of T-Cells by SNAs

Antigen-specific T-cell responses depend upon the interaction between activated DCs and T-cells; the quality of this interaction and subsequent T-cell response are dependent upon the concerted presentation of antigen and expression of co-stimulatory markers by DCs upon vaccination.¹⁷ The kinetics of the parallel pathways of presentation of SNA-delivered OVA1 and the expression of the co-stimulatory markers CD40 and CD86 where therefore compared in BMDCs. Following the treatment of BMDCs with SNAs for 30 minutes (5 μM in OVA1 and CpG) and subsequent washing to remove SNAs from cell culture medium, cells were re-suspended and incubated in fresh medium for up to 48 hours. Although the maximum expression of CD40 and CD86 took place approximately 24 hours after treatment for all three SNA structures (FIGS. 4A), notably the time at which OVA1 presentation was maximized was different among the SNAs (approximately 16 hours for SNA E, and approximately 20 hours for SNAs A and approximately 24 hours for H, FIG. 4A). A major consequence of the slower kinetics of antigen presentation induced by SNAs A and H (compared to SNA E), due to the processing and dissociation of OVA1 from these SNA structures, was greater overlap in time where DCs present both antigen and co-stimulatory markers. Importantly, the kinetic data for SNA H showed synchronization of maximized antigen presentation and co-stimulatory marker expression (FIG. 4A). Taken together with the superior ability of SNA H to co-deliver CpG and peptide to DCs, these data showed that SNA H may be ideal for the priming of antigen-specific T-cells.

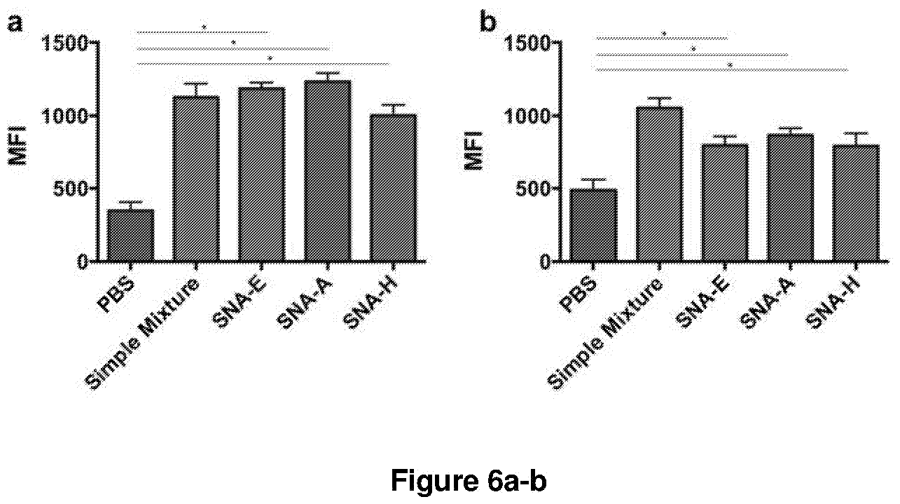

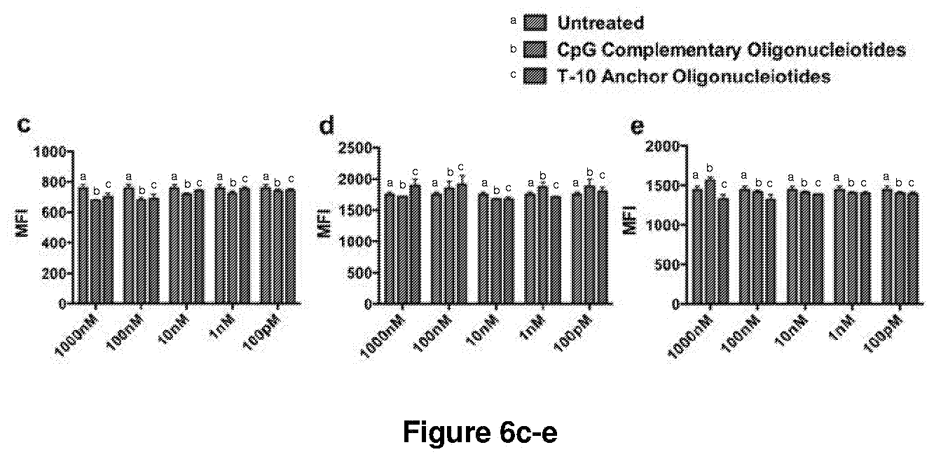

Immunization by subcutaneous injection of SNAs resulted in DC activation and antigen presentation in vivo. In all three SNA designs, the DLNs of immunized C57BL/6 mice swelled and showed increased cellularity (16 hours following immunization), compared to those of mice immunized with a mixture of CpG and OVA1 (FIG. 4B). CD80 expression on CD11c⁺ DCs in DLNs was higher for SNAs A and H than for SNA E or a mixture of CpG and OVA1 (FIG. 4C), while expression levels of CD86 and CD40 were comparable across all treatment groups (FIG. 6a-b).

Next, the ability of DCs activated by SNAs in vivo to cross-prime CD8⁺ T-cells was examined. DCs from the DLN were harvested from immunized mice and co-cultured with OT1 CD8+ T cells for 2 days ex vivo. The secretion of pro-inflammatory cytokines (IL-12p70, IL-1α, IL-6 and TNF-α) was highly dependent on SNA structure. Although each SNA structure (E, A, H) led to greater levels of cytokine secretion than that for mixtures of CpG and OVA1 (FIG. 4D-4G), SNAs H and A were superior to SNA E in stimulating the secretion of IL-1a, IL-6, and TNF-α by OVA1-specific T-cells. In addition, ELISPOT was used to examine the number of IFN-γ-secreting- T-cells generated by co-culturing with DCs from immunized mice. The DCs extracted from SNA H- and SNA A-immunized mice showed a greater ability to induce IFN-γ production from OT1 CD8+ T cells, as compared to those extracted from SNA E-immunized mice (FIGS. 4H and 6e). Importantly, vaccination with oligonucleotides conjugated to OVA1 not formulated as SNAs had negligible effect on non-antigen-specific DC-activation (FIG. 6c-e). These observations demonstrate that differences in SNA structure ultimately lead to substantial differences in the quality of antigen-specific T-cell responses.

### Antigen-Specific CTL Responses Generated by Vaccination With SNAs

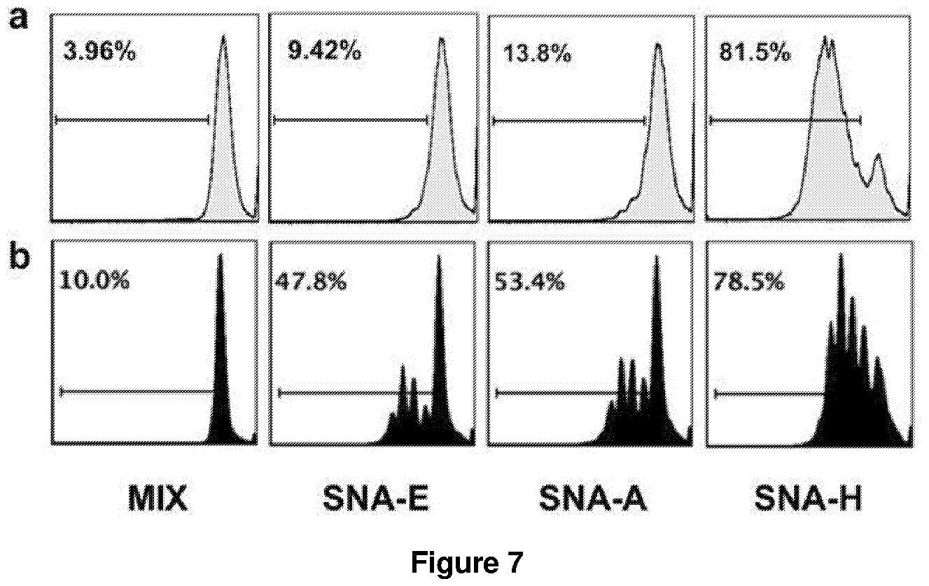

The quality of antigen-specific CTL responses induced by the vaccination of immunocompetent mice (C57BL/6) by SNA structures E, A, H and for comparison, mixtures of CpG and antigen, were compared. The comparison of SNA structures for three different antigens was performed: OVA1 (FIGS. 5A-D and 7a), E6 (FIG. 5E-H, J), and gp100 (FIG. 7b).^{25,26} It was found that the influence of SNA structure on raising antigen-specific T-cells is not limited to OVA or restricted by the selection of antigen. The data of FIG. 5 show that SNA structures were superior to mixtures of CpG and peptide antigen, at generating cytotoxic and memory phenotypes in antigen-specific CD8⁺ T-cells in vivo through the incorporation of OVA1 (FIGS. 5A-B 4A-B) and E6 (FIGS. 5E-F). The effector function of antigen-specific CD8⁺ T-cells raised in immunized mice, as measured by IFN-γ secretion via both ELISPOT assay and flow cytometry, were significantly increased for mice vaccinated with SNAs A and H, for both OVA1 and E6 (FIG. 5C-D, G-H). Vaccination with mixtures of CpG and peptide yielded negligible numbers of IFN-γ secreting T-cells, as did vaccination with SNA E for E6 (FIG. 5G,H).

For T-cells raised by SNAs formulated with OVA1, SNA H led to the greatest efficacy in killing target cells (EG.7-OVA) in a dose-dependent fashion (FIG. 51). Furthermore, the killing of target cells showed a clear dependence on SNA structure, following the order of H>A>E>mixture of CpG and OVA1. For the targeted killing of TC-1 cells, vaccinations with SNA H and A with E6 led to comparable CTL performances that were far superior to that induced by SNA E or a mixture of CpG and E6. These data indicated that the structure of SNA H, by way of the advantages in its interaction with DCs, ultimately leads to superior antigen-specific T-cell responses in vivo. The effect of SNA structure on CTL activity was however more emphatic for E6 than for OVA1. Whether the differences observed between these two antigen systems is driven primarily by the intrinsic immunogenicity of the E6 and OVA1 antigens, or by the influence of the peptide antigens on the properties of SNAs, warrants further investigation. Taken together, these experiments indicated the broad applicability of SNA structures, and in particular SNA H, in raising immune responses to different tumor-specific antigens and ultimately their use in cancer immunotherapy.

### SNA Structure-Dependent Anti-Tumor Immune Responses

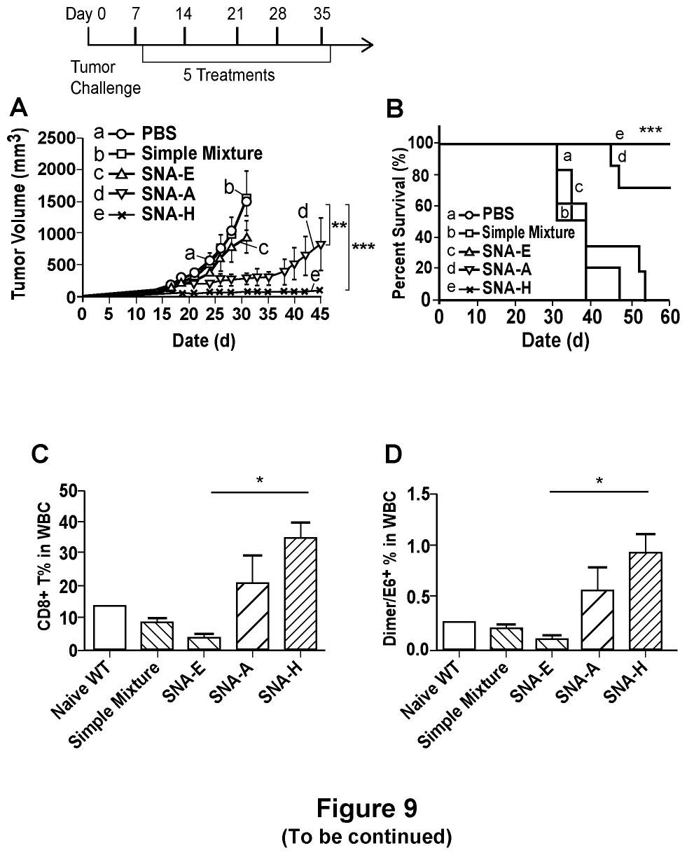

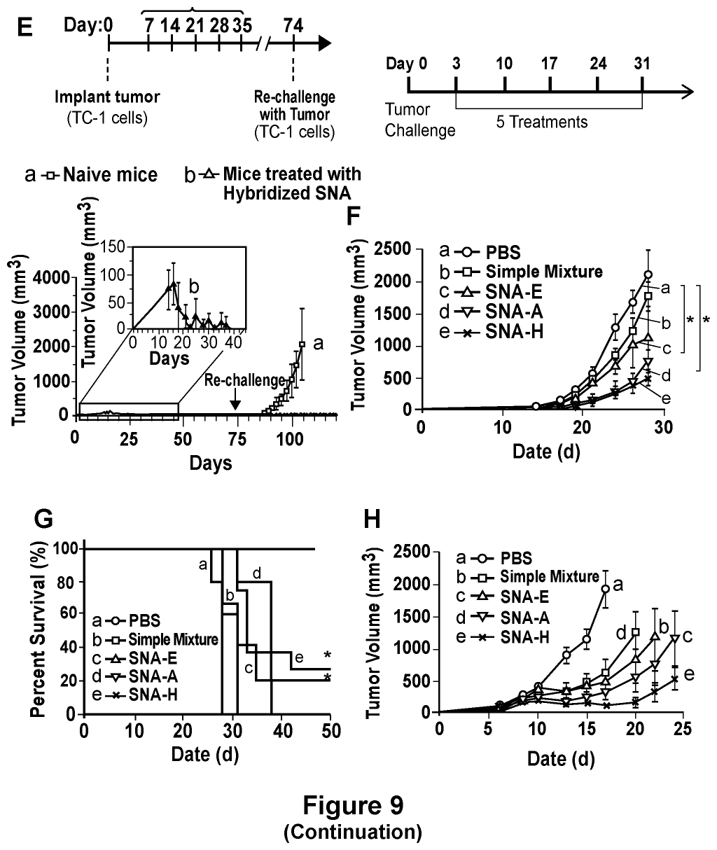

To evaluate SNA structures as potential immunotherapeutic agents for cancer, three well-established tumor-bearing mouse models were tested with SNAs. TC-1 tumors were generated by subcutaneous implantation of TC-1 cells in the flanks of C57BL/6 mice and then allowing them to grow to approximately 50mm³ prior to treatment with SNA structures E, A, and H, each formulated with the E6 antigen (7-10 mice per group). Additional groups for untreated mice and treatment with a mixture of CpG and E6 peptide served as control and reference groups. Treatment consisted of an initial vaccination followed by four boosts, with 7 days in between each boost (FIG. 9A, Scheme). Treatment with SNA H strikingly led to tumor regression and survival of 100% of the animals in the group through 60 days (FIG. 9A-B). In contrast, treatment with mixtures of CpG and E6 or SNA E failed to deliver significant improvements in tumor burden or survival over the untreated group, suggesting that the antitumor efficacy of SNAs is highly dependent upon the SNA structure. Within the SNA H treatment group, 30% of the animals were in a tumor-free condition till 72 days. These tumor-free mice were subsequently re-challenged (on day 72) with an inoculation of fresh TC-1 cells into the flank opposing the initial tumor site but were not given any additional therapy. These mice rejected the implanted TC-1 cells, while tumor growth was aggressive in a reference group (naïve mice that had received no prior vaccination) (FIG. 9E). This observation showed that the immunological memory generated by the treatment with SNA H leads to long term tumor protection. The growth of TC-1 tumors was also significantly inhibited by treatment with SNA A (FIG. 9A); 70% of the animals treated with SNA A survived through 60 days.

The efficacy of SNA H and SNA E in tumor inhibition and survival was consistent with the tumor antigen-specific CD8⁺ T-cell responses raised by these vaccines. The percentages of overall CD8⁺ T-cells and E6-specific CD8⁺ T cells within WBC were highest for peripheral blood sampled (on day 40) from animals treated with SNA H and SNA A (34.8% and 20.7% respectively, for CD8⁺ T-cells; and 0.9% and 0.6% respectively, for E6-specific CD8+ T-cells). These percentages were significantly lower for the other treatment groups (3.5% and 8.4% for CD8⁺ T-cells in the SNA E and PBS-treated groups, respectively; 0.1% and 0.2% for E6-specific CD8⁺ T-cells) (FIG. 9C-D).

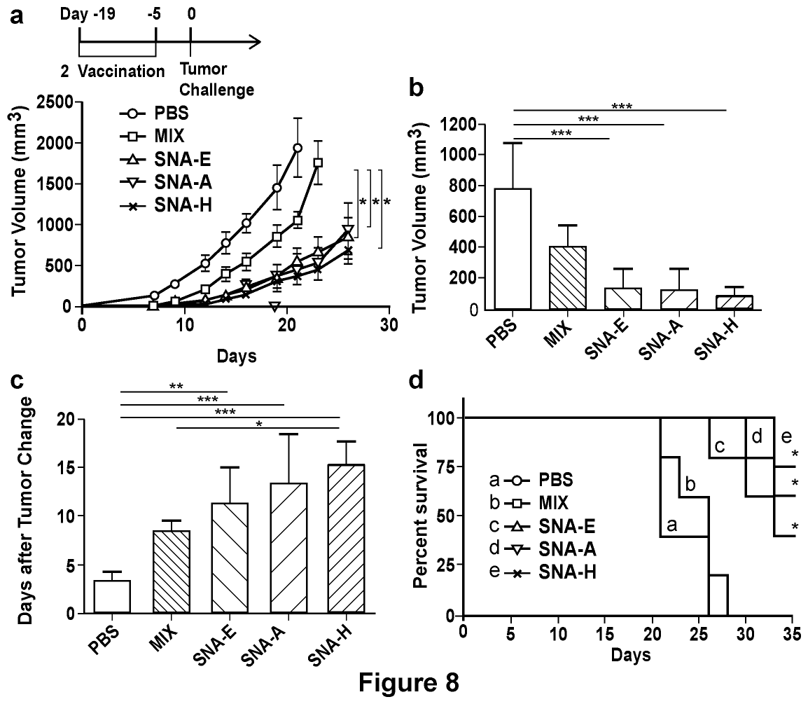

The quality of anti-tumor immune responses in mice bearing LLC-OVA tumors and EG-7-OVA were also found to be highly SNA structure-dependent. Treatment with SNAs H and A functionalized with OVA peptide resulted in the best outcomes in tumor growth inhibition and animal survival; 80% of animals in these groups survive through day 31, a time point at which 100% of the animals had perished in groups of animals that were untreated or treated with a mixture of CpG and OVA (FIG. 9F-G). The use of SNAs in prophylactic vaccination was capable of delaying LLC-OVA tumor initiation and growth. Animals were vaccinated 21 and 7 days (primary injection and boost, respectively) prior to implantation of LLC-OVA cells. Each SNA structure was superior to a mixture of CpG and OVA peptide in delaying the initiation of tumor growth and prolonging survival (FIG. 8a-d). Prophylactic vaccination with SNA H led to the best outcomes, resulting in a 15 day delay in tumor initiation, longer than that observed for vaccination with SNA A (13 days) or E (11 days) (FIG. 8c). With EG-7-OVA tumor treatment, the same dosing and treatment plan was used as that used in the treatment of mouse models of LLC1-OVA. Treatment with SNA H functionalized with OVA peptide resulted in the best outcomes in tumor growth inhibition (FIG. 9H), while SNA-E and A led to outcomes comparable to those for mixtures of antigen and CpG.

The examination of the effects of SNA structure on three different tumor models revealed that treatment with SNA H leads to the best outcomes in tumor burden and animal survival. Treatment with SNA A leads to significantly better outcomes than those for SNA E or mixtures of CpG and antigen; in the LLC-OVA model, treatments with SNA A and H lead to comparable outcomes while EG-7-OVA model revealed the best outcomes for SNA H. These results also showed differences in the efficacy of SNA vaccination and the dependence on SNA structure between the TC-1 (E6), LLC (OVA) and EG7 (OVA) models, particularly in the elimination of TC-1 tumors upon treatment with SNA H. These differences are likely due to the immunogenicity of the antigens used (E6 and OVA1) and the aggressiveness of the cells used to generate the tumor models. These tumor models have been used to illustrate the anti-tumor activity of vaccines using other materials (e.g., polymer-based delivery of antigen and adjuvant). The study of SNAs in the present disclosure, however, showed efficacy using structures composed of FDA-approved classes of materials (i.e., liposomes, oligonucleotides) and provides a way to avoid the chronic liver toxicity that may arise from the use of polymeric materials^{27,28}.

### CONCLUSION

This study of compositionally equivalent yet structurally distinct SNAs has determined that differences in SNA structure can lead to major improvements in raising cellular immune responses and outcomes in anti-tumor immunotherapy. A key lesson from this study is that even within a single class of materials, the way in which adjuvant molecules and tumor-associated antigens are structured within a vaccine can profoundly influence the activation of immune responses. Numerous comparisons of uptake and intracellular trafficking (FIGS. 1 and 3), DC activation (FIG. 4), T-cell activation (FIG. 5), and therapeutic outcomes in vivo (FIG. 9) showed the inability of mixtures of CpG and peptide antigen to boost effective immune responses, while consistently resulted in the ability of SNA structures to invoke responses in a manner clearly dependent upon how the SNA structures incorporate antigen and adjuvant molecules (H>A>E). These differences are emphatic in the interaction of SNAs with DCs, by controlling the co-delivery of CpG and peptide, the subcellular trafficking and retention of peptides within individual cells, and synchronizing the kinetics of processing of CpG and antigen; these differences ultimately drive the quality of the effector function of antigen-specific killing of tumor cells in vivo and range from essentially ineffective to curative. Indeed, the modularity of SNAs has led to the identification of SNA H as superior among the structures studied. Given the scalability and clinical relevance of SNAs, this work provides a route to creating effective vaccines for many conditions.

It is to be understood that the foregoing description is exemplary and explanatory only and are not restrictive of any subject matter claimed. In this application, the use of the singular includes the plural unless specifically stated otherwise; the singular forms “a,” “an,” and “the” include plural referents unless the context clearly dictates otherwise. The term “comprising,” the term “having,” the term “including,” and variations of these words are intended to be open-ended and mean that there may be additional elements other than the listed elements.

### REFERENCES

1 Coulie, P. G., Van den Eynde, B. J., Van Der Bruggen, P. & Boon, T. Tumour antigens recognized by T lymphocytes: at the core of cancer immunotherapy. Nature Reviews Cancer 14, 135 (2014).

2 Khalil, D. N., Smith, E. L., Brentjens, R. J. & Wolchok, J. D. The future of cancer treatment: immunomodulation, CARs and combination immunotherapy. Nature reviews Clinical oncology 13, 273 (2016).

3 Irvine, D. J., Swartz, M. A. & Szeto, G. L. Engineering synthetic vaccines using cues from natural immunity. Nature materials 12, 978 (2013).

4 Gulley, J. L. et al. Avelumab (MSB0010718C), an anti-PD-L1 antibody, in advanced NSCLC patients: A phase 1 b, open-label expansion trial in patients progressing after platinum-based chemotherapy. Journal of Clinical Oncology 33, 8034-8034, doi:10.1200/jco.2015.33.15_suppl.8034 (2015).

5 Patnaik, A. et al. Phase 1 study of pembrolizumab (pembro; MK-3475) plus ipilimumab (IPI) as second-line therapy for advanced non-small cell lung cancer (NSCLC): KEYNOTE-021 cohort D. Journal of Clinical Oncology 33, 8011-8011, doi:10.1200/jco.2015.33.15_suppl.8011 (2015).

6 Weber, J. S. et al. Nivolumab versus chemotherapy in patients with advanced melanoma who progressed after anti-CTLA-4 treatment (CheckMate 037): a randomised, controlled, open-label, phase 3 trial. The Lancet Oncology 16, 375-384, doi:https://doi.org/10.1016/S1470-2045(15)70076-8 (2015).

7 Boutros, C. et al. Safety profiles of anti-CTLA-4 and anti-PD-1 antibodies alone and in combination. Nature Reviews Clinical Oncology 13, 473, (2016).

8 Naidoo, J. et al. Toxicities of the anti-PD-1 and anti-PD-L1 immune checkpoint antibodies. Annals of Oncology 26, 2375-2391 (2015).

9 Tiwari, G. et al. Drug delivery systems: An updated review. International journal of pharmaceutical investigation 2, 2 (2012).

10 Blanco, E., Shen, H. & Ferrari, M. Principles of nanoparticle design for overcoming biological barriers to drug delivery. Nature biotechnology 33, 941 (2015).

11 Frey, S., Castro, A., Arsiwala, A. & Kane, R. S. Bionanotechnology for vaccine design. Current opinion in biotechnology 52, 80-88 (2018).

12 Irvine, D. J., Hanson, M. C., Rakhra, K. & Tokatlian, T. Synthetic nanoparticles for vaccines and immunotherapy. Chemical reviews 115, 11109-11146 (2015).

13 Couvreur, P. Nanoparticles in drug delivery: past, present and future. Advanced drug delivery reviews 65, 21-23 (2013).

14 Kemp, J. A., Shim, M. S., Heo, C. Y. & Kwon, Y. J. “Combo” nanomedicine: Co-delivery of multi-modal therapeutics for efficient, targeted, and safe cancer therapy. Advanced Drug Delivery Reviews 98, 3-18, doi:https://doi.org/10.1016/j.addr.2015.10.019 (2016).

15 Gu, L. & Mooney, D. J. Biomaterials and emerging anticancer therapeutics: engineering the microenvironment. Nature Reviews Cancer 16, 56 (2016).

16 Koshy, S. T. & Mooney, D. J. Biomaterials for enhancing anti-cancer immunity. Current opinion in biotechnology 40, 1-8 (2016).

17 Tzeng, A. et al. Temporally programmed CD8a+DC activation enhances combination cancer immunotherapy. Cell reports 17, 2503-2511 (2016).

18 Rincon-Restrepo, M. et al. Vaccine nanocarriers: Coupling intracellular pathways and cellular biodistribution to control CD4 vs CD8 T cell responses. Biomaterials 132, 48-58 (2017).

19 Rosi, N. L. et al. Oligonucleotide-modified gold nanoparticles for intracellular gene regulation. Science 312, 1027-1030 (2006).

20 Zheng, D. et al. Topical delivery of siRNA-based spherical nucleic acid nanoparticle conjugates for gene regulation. Proceedings of the National Academy of Sciences 109, 11975-11980 (2012).

21 Kapadia, C. H., Melamed, J. R. & Day, E. S. Spherical Nucleic Acid Nanoparticles: Therapeutic Potential. BioDrugs, 1-13 (2018).

22 Jensen, S. A. et al. Spherical nucleic acid nanoparticle conjugates as an RNAi-based therapy for glioblastoma. Science translational medicine 5, 209ra152-209ra152 (2013).

23 Radovic-Moreno, A. F. et al. Immunomodulatory spherical nucleic acids. Proceedings of the National Academy of Sciences 112, 3892-3897 (2015).

24 Skakuj, K. et al. Conjugation Chemistry-Dependent T-Cell Activation with Spherical Nucleic Acids. Journal of the American Chemical Society 140, 1227-1230, (2018).

25 Luo, M. et al. A STING-activating nanovaccine for cancer immunotherapy. Nature nanotechnology 12, 648 (2017).

26 Bakker, A. B. H. Melanocyte lineage-specific antigen gp100 in T cell-mediated immunotherapy of melanoma, (1996).

27 Li, W. A. & Mooney, D. J. Materials based tumor immunotherapy vaccines. Current Opinion in Immunology 25, 238-245, (2013).

28 Li, L. et al. Polymer- and lipid-based nanoparticle therapeutics for the treatment of liver diseases. Nano Today 5, 296-312, (2010).

What is claimed is:

1. A method comprising:
treating a population of antigen presenting cells with a spherical nucleic acid (SNA) comprising a nanoparticle, an antigen, and an adjuvant; and
determining a time at which the population of antigen presenting cells presents a maximal signal that is indicative of antigen presentation by the antigen presenting cells and a time at which the population of antigen presenting cells presents a maximal co-stimulatory signal due to the adjuvant.

2. A method of selecting a spherical nucleic acid (SNA) for increased ability to activate antigen presenting cells, comprising:
generating a first SNA comprising a nanoparticle, an antigen, and an adjuvant and a second SNA comprising nanoparticle, an antigen, and an adjuvant;
treating a first population of antigen presenting cells with the first SNA and treating a second population of antigen presenting cells with the second SNA;
determining a time at which the first population of antigen presenting cells presents a maximal signal that is indicative of antigen presentation and a time at which the first population of antigen presenting cells presents a maximal co-stimulatory signal due to the adjuvant;
determining a time at which the second population of antigen presenting cells presents a maximal signal that is indicative of antigen presentation and a time at which the second population of antigen presenting cells presents a maximal co-stimulatory signal due to the adjuvant; and
selecting as the SNA for which time to achieve maximal signal for antigen presentation is the same as or about the same as time to achieve maximal co-stimulatory signal.

3. A spherical nucleic acid (SNA) comprising a nanoparticle, an adjuvant, and an antigen, wherein:
the adjuvant comprises an oligonucleotide comprising an immunostimulatory nucleotide sequence and an associative moiety that allows association of the immunostimulatory sequence with the nanoparticle; and
the antigen is attached to the nanoparticle through a linker.

4. The SNA of claim 3, wherein the immunostimulatory nucleotide sequence is a toll-like receptor (TLR) agonist.

5. The SNA of claim 4, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

6. The SNA of any one of claims 3-5, wherein the immunostimulatory nucleotide sequence comprises a CpG nucleotide sequence.

7. The SNA of any one of claims 3-6, wherein the linker is a carbamate alkylene disulfide linker.

8. The SNA of claim 7, wherein the antigen is attached to the nanoparticle through the linker according to Antigen-NH—C(O)—O-C₂₋₅alkylene-S—S-C₂₋₇alkylene, or Antigen-NH—C(O)—O—CH₂—Ar—S—S-C₂₋₇alkylene, wherein Ar comprises a meta- or para-substituted phenyl.

9. The SNA of claim 8, wherein the antigen is attached to the nanoparticle through the linker according to Antigen-NH—C(O)—O-C₂₋₄alkylene-C(W)(X)—S—S—CH(Y)(Z)C₂₋₆alkylene, and W and X, Y and Z are each independently H, Me, Et, or iPr.

10. The SNA of claim 8, wherein the antigen is attached to the nanoparticle through the linker according to Antigen-NH—C(O)—O—CH₂—Ar—S—S—CX(Y)C₂₋₆alkylene, and X and Y are each independently Me, Et, or iPr.

11. The SNA of any one of claims 3-6, wherein the linker is an amide alkylene disulfide linker.

12. The SNA of claim 11, wherein the antigen is attached to the nanoparticle through the linker according to Antigen-NH—C(O)-C₂₋₅alkylene-S—S-C₂₋₇alkylene.

13. The SNA of claim 12, wherein the antigen is attached to the nanoparticle through the linker according to Antigen-NH—C(O)—C(W)(X)C₂₋₄alkylene-S—S—CH(Y)(Z)C₂₋₆alkylene, and W and X, Y and Z are each independently H, Me, Et, or iPr.

14. The SNA of any one of claims 3-6, wherein the linker is a amide alkylene thio-succinimidyl linker.

15. The SNA of claim 14, wherein the antigen is attached to the nanoparticle through the linker according to Antigen-NH—C(O)-C₂₋₄alkylene-N-succinimidyl-S-C₂₋₆alkylene.

16. The SNA of any one of claims 3-15, wherein the antigen is a tumor associated antigen, a tumor specific antigen, a neo-antigen.

17. The SNA of claim 16, wherein the antigen is OVA1, MSLN, P53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, or human papillomavirus (HPV) E7 nuclear protein.

18. The SNA of any one of claims 3-17, wherein the nanoparticle is a liposome.

19. The SNA of claim 18, wherein the liposome comprises a lipid selected from the group consisting of 1,2-dioleoyl-sn-glycero-3-phosphocholine (DOPC), 1,2-dimyristoyl-sn -phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho -(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

20. The SNA of any one of claims 3-19, wherein the associative moiety is tocopherol, cholesterol, 1,2-distearoyl-sn-glycero-3-phospho-(1′-rac-glycerol) (DSPG), 1,2-dioleoyl-sn -glycero-3-phospho-(1′-rac-glycerol) (DOPG), 1,2-di-(9Z-octadecenoyl)-sn-glycero -3-phosphoethanolamine (DOPE), or 1,2-dihexadecanoyl-sn-glycero-3-phosphoethanolamine (DPPE).

21. The SNA of any one of claims 3-20, wherein the adjuvant comprises RNA or DNA.

22. The SNA of any one of claims 3-21, further comprising an additional oligonucleotide.

23. The SNA of claim 22, wherein the additional oligonucleotide comprises RNA or DNA.

24. The SNA of claim 23, wherein said RNA is a non-coding RNA.

25. The SNA of claim 24, wherein said non-coding RNA is an inhibitory RNA (RNAi).

26. The SNA of claim 24 or claim 25, wherein the RNAi is selected from the group consisting of a small inhibitory RNA (siRNA), a single-stranded RNA (ssRNA) that forms a triplex with double stranded DNA, and a ribozyme.

27. The SNA of claim 24 or claim 25, wherein the RNA is a microRNA.

28. The SNA of claim 23, wherein said DNA is antisense-DNA.

29. The SNA of any one of claims 3-28, wherein the nanoparticle has a diameter of 50 nanometers or less.

30. The SNA of any one of claims 3-29 comprising about 10 to about 80 double stranded oligonucleotides.

31. The SNA of claim 30 comprising 75 double stranded oligonucleotides.

32. The method of claim 1, wherein the SNA is the SNA of any one of claims 3-31.

33. The method of claim 2, wherein the first SNA and/or the second SNA is independently the SNA of any one of claims 3-31.

34. A composition comprising the SNA obtained by the method of claim 1 or claim 2 in a pharmaceutically acceptable carrier.

35. The composition of claim 34, wherein the composition is capable of generating an immune response in an individual upon administration to the individual.

36. The composition of claim 35, wherein the immune response comprises antibody generation or a protective immune response.

37. A vaccine comprising the composition of any one of claims 34-36, and an adjuvant.

38. The composition of claim 35, wherein the immune response is a neutralizing antibody response or a protective antibody response.

39. A method of producing an immune response to cancer in an individual, comprising administering to the individual an effective amount of the composition of claims 34-36, or the vaccine of claim 37, thereby producing an immune response to cancer in the individual.

40. A method of inhibiting expression of a gene comprising hybridizing a polynucleotide encoding the gene with one or more oligonucleotides complementary to all or a portion of the polynucleotide, the oligonucleotide being the additional oligonucleotide of the SNA of any one of claims 22-31, wherein hybridizing between the polynucleotide and the oligonucleotide occurs over a length of the polynucleotide with a degree of complementarity sufficient to inhibit expression of the gene product.

41. The method of claim 40 wherein expression of the gene product is inhibited in vivo.

42. The method of claim 40 wherein expression of the gene product is inhibited in vitro.

43. A method for up-regulating activity of a toll-like receptor (TLR) comprising contacting a cell having the TLR with a SNA of any one of claims 3-31 or the SNA obtained by the method of claim 1 or claim 2.

44. The method of claim 43 wherein the adjuvant comprises a TLR agonist.

45. The method of claim 43 or claim 44 wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

46. The method of any one of claims 43-45 which is performed in vitro.

47. The method of any one of claims 43-45 which is performed in vivo.

48. The method of any one of claims 43-47, wherein the cell is an antigen presenting cell (APC).

49. The method of claim 48, wherein the APC is a dendritic cell.

50. The method of claim 48, wherein the cell is a leukocyte.

51. The method of claim 50, wherein the leukocyte is a phagocyte, an innate lymphoid cell, a mast cell, an eosinophil, a basophil, a natural killer (NK) cell, a T cell, or a B cell.

52. The method of claim 51, wherein the phagocyte is a macrophage, a neutrophil, or a dendritic cell.

53. A method of immunizing an individual against cancer comprising administering to the individual an effective amount of the composition of any one of claims 34-36, thereby immunizing the individual against cancer.

54. The method of claim 53, wherein the composition is a cancer vaccine.

55. The method of claim 53 or 54, wherein the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.

56. The method of claim 1 or claim 2, wherein the antigen presenting cells is a dendritic cell or a lymphocyte.


SEQUENCE LISTING (13 sequences)

SEQ ID NO: 1 - protein, 9 aa, Artificial Sequence
  Synthetic polypeptide
CSIINFEKL

SEQ ID NO: 2 - protein, 10 aa, Artificial Sequence
  Synthetic polypeptide
CKVPRNQDWL

SEQ ID NO: 3 - protein, 10 aa, Artificial Sequence
  Synthetic polypeptide
VYDFAFRDLC

SEQ ID NO: 4 - DNA, 20 bp, Artificial Sequence
  Synthetic polynucleotide
  misc_feature (20)..(20): (Sp18)2 Cholesterol
tccatgacgttcctgacgtt

SEQ ID NO: 5 - DNA, 22 bp, Artificial Sequence
  Synthetic polynucleotide
  misc_feature (20)..(20): (Sp18)2
tccatgacgttcctgacgtttt

SEQ ID NO: 6 - DNA, 20 bp, Artificial Sequence
  Synthetic polynucleotide
  misc_feature (20)..(20): Cy5- (Sp18)2-Cholesterol
tccatgacgttcctgacgtt

SEQ ID NO: 7 - DNA, 22 bp, Artificial Sequence
  Synthetic polynucleotide
  misc_feature (20)..(20): (Sp18)2
tccatgacgttcctgacgtttt

SEQ ID NO: 8 - DNA, 20 bp, Artificial Sequence
  Synthetic polynucleotide
  misc_feature (20)..(20): SH
aacgtcaggaacgtcatgga

SEQ ID NO: 9 - DNA, 10 bp, Artificial Sequence
  Synthetic polynucleotide
  misc_feature (1)..(1): SH
  misc_feature (1)..(10): Cholesterol
tttttttttt

SEQ ID NO: 10 - protein, 8 aa, Artificial Sequence
  Synthetic polypeptide
SIINFEKL

SEQ ID NO: 11 - protein, 12 aa, Artificial Sequence
  Synthetic polypeptide
  MISC_FEATURE (4)..(4): Alpha-NH
TMRSSIINFEKL

SEQ ID NO: 12 - protein, 10 aa, Artificial Sequence
  Synthetic polypeptide
CKVPRNQDWL

SEQ ID NO: 13 - protein, 10 aa, Artificial Sequence
  Synthetic polypeptide
VYDFAFRDLC